(19) United States
(12) Patent Application Publication
(10) Pub. No.: US 2012/0277283 A1
(43) Pub. Date: Nov. 1, 2012

(54) Localized Delivery of Gold Nanoparticles for Therapeutic and Diagnostic Applications

(76) Inventors: Chad A. Mirkin, Wilmette, IL (US); Reed A. Omary, Wilmette, IL (US); Aaron Eifler, Chicago, IL (US); Samdeep K. Mouli, Chicago, IL (US); Kaylin McMahon, Chicago, IL (US); Andrew Larson, Kildeer, IL (US); C.S. Thaxton, Chicago, IL (US)

(21) Appl. No.: 13/388,630

(22) PCT Filed: Aug. 4, 2010
(86) PCT No.: PCT/US2010/044453

§ 371 Date: Jul. 13, 2012

Related U.S. Application Data
(60) Provisional application No. 61/231,214, filed on Aug. 4, 2009.
(60) Provisional application No. 61/295,640, filed on Jan. 15, 2010.
(60) Provisional application No. 61/296,361, filed on Jan. 19, 2010.
(60) Provisional application No. 61/314,145, filed on Mar. 15, 2010.

(51) Int. Cl. A61K 31/7088; A61P 35/00

(52) U.S. Cl. 514/44 A; 514/44 R

(57) ABSTRACT

The present invention is directed to compositions and methods of localized delivery of a functionalized nanoparticle.

### CROSS-REFERENCE TO RELATED APPLICATIONS

This application claims the priority benefit under 35 U.S.C. §119(e) of U.S. Provisional Application No. 61/231,214, filed Aug. 4, 2009, U.S. Provisional Application No. 61/314,145, filed Mar. 15, 2010, U.S. Provisional Application No. 61/296,361, filed Jan. 19, 2010, and U.S. Provisional Application No. 61/295,640, filed Jan. 15, 2010, the disclosures of which are incorporated herein by reference in their entirety.

### STATEMENT OF GOVERNMENT INTEREST

This invention was made with government support under Grant Number 5DP1 OD000285, awarded by the National Institutes of Health (NIH), and Grant Number N5U54 CA119341, awarded by the NIH (NCI/CCNE). The government has certain rights in the invention.

### FIELD OF THE INVENTION

The present invention is directed to compositions and methods of localized delivery of a functionalized nanoparticle.

### BACKGROUND OF THE INVENTION

Nanoparticle chemistry has been shown to be extremely promising in a variety of applications including medical therapy. Gold nanoparticles (AuNPs), for example, have been shown to be non-toxic and when surface functionalized with polynucleotides (i.e. by covalently attaching polynucleotides to the surface of AuNPs), are able to be taken up by a variety of cell types with approximately 99% efficiency. Also, the polynucleotides attached to the gold nanoparticle have been shown to be extremely stable. Thus, gold nanoparticles can be used to transfect cells with polynucleotides and represent a non-toxic and efficient way to introduce polynucleotides into cells for protein knockdown.

Intraarterial drug delivery, pioneered and perfected by the field of interventional radiology (IR), has been used extensively in the minimally invasive treatment of a wide variety of diseases including solid tumors. IR physicians are able to catheterize the blood supply directly feeding a solid tumor and deliver relatively high doses of chemotherapeutics while limiting the systemic side effects of such drugs.

Cancer is one of the leading causes of death in this country. In the past few decades, major progress has been made in the treatment strategies for this disorder. However, there still remains a significant morbidity and mortality associated with cancer. As the fourth leading cause of cancer related mortality in the United States [American Cancer Society. Cancer Facts & Figures 2008. (2008)], pancreatic cancer carries with it a dismal prognosis. Nearly 99% of those diagnosed with pancreatic cancer will die of their disease, with a median survival of 6 months and 5-year survival of less than 5% across all stages [Ries et al., SEER Cancer Statistics Review, 1975-2005. (2008)]. Pancreatic cancer remains resistant to nearly all available treatments [Feldmann et al., J Mol Diagn 10: 111-22. (2008)] with surgical resection remaining the only potentially curative measure [Ghaneh et al., Gut 56: 1134-52. (2007)]. Resection, however, is possible in less than 20% of cases and of those patients, median 5-year survival is 12% [Garcea et al., Journal of the Pancreas 9: 99-132. (2008)]. Although gemcitabine, paired with other cytotoxic agents, is the front line treatment for advanced inoperable pancreatic cancer, median survival is still <7 months [Abou-Alfa et al., J Clin Oncol 24: 4441-7. (2006)].

Given these grim statistics, there is a clear need to develop innovative approaches to treat pancreatic cancer. Interventional radiology therapies directed towards hepatic malignancies, such as chemoembolization, have gained widespread acceptance because of their ability to improve survival and/or induce a tumor response that can be confirmed by post-treatment imaging [Llovet et al., Lancet 359: 1734-9. (2002)]. Preliminary studies of arterial infusion chemotherapy for advanced pancreatic cancer [Homma et al., Cancer 89: 303-13. (2000)] show that this method of drug delivery may provide significant gains in 1-year survival [Miyanishi et al., Jpn J Clin Oncol 38: 268-74. (2008)].

There are a number of molecular targets elucidated for pancreatic cancer. For instance, nearly 100% of pancreatic adenocarcinomas have altered KRAS expression [Bardeesy et al., Nat Rev Cancer 2: 897-909. (2002)]. In addition, 75% of tumors express a mutant p53 tumor suppressor gene [Li et al., The Lancet 363: 1049-1057. (2004)]. More recently, survivin, a member of the apoptosis inhibiting protein family, has been found to be a central regulator in the immortalization of cancer cells, is differentially expressed in cancer cells versus normal cells, and is a central target for cancer cells with mutations in a number of key regulatory pathways, including p53 [Alfieri, Nat Rev Cancer 8: 61-70. (2008)]. As would be expected, survivin is an evolving and exciting molecular target for pancreatic cancer [Hamacher et al., Mol Cancer 7: 64. (2008)].

Introduction of genetic material into cells and tissues to control gene expression holds significant promise for therapeutic application [Lebedeva et al., Annu Rev Pharmacol Toxicol 41: 403-19. (2001)]. Developing nucleic acids, including short interfering RNA (siRNA) and antisense DNA species, into viable therapeutic agents has faced challenges with regard to: 1) stable cellular transfection; 2) entry into diverse cell types; 3) toxicity; and 4) efficacy [Lebedeva et al., Annu Rev Pharmacol Toxicol 41: 403-19. (2001)]. To overcome these shortcomings, nanoparticle conjugates have been investigated to introduce antisense DNA and siRNA into cells and tissues. Gold nanoparticles densely functionalized with DNA have been successfully used as antisense agents to suppress gene expression in vitro without the use of transfection reagents [Rosi et al., Science. 312: 1027-30. (2006)]. Gold is considered to be biocompatible and safe for in vivo use [Connor et al., Small 1: 325-7. (2005)].

RNA inhibition (RNAi) works though complementary Watson-Crick base pairing of a guide strand to the messenger RNA (mRNA) that is to be inhibited (the target strand) reducing the amount of protein translated from the target mRNA (termed “protein knockdown”). In almost all cancers, upregulated proteins give cancer cells the ability to avoid apoptosis and proliferate when they should not.

### SUMMARY OF THE INVENTION

Described herein is a nanoparticle composition comprising a polynucleotide-functionalized nanoparticle and an embolic agent. The nanoparticle composition is useful for localized delivery to a site of pathogenesis, increased retention time and genetic regulation. The composition described herein enters cells without transfection agents and is resistant to degradation in a manner that enhances knockdown activity compared to conventional polymer carriers. Also, the embolic agent as described herein is shown to increase the retention time of the composition at the desired site of delivery, thereby increasing the effectiveness of the composition. Finally, localized delivery approaches could incorporate any technique to guide treatment and verify delivery to a specific site as well as take advantage of novel molecular targeting of intracellular mechanisms specific to a specific cell.

The delivery of polynucleotide-functionalized nanoparticles (PN-NPs) to the site of disease is a desirable modality of therapy. Intravenous (IV) delivery, however, is hampered by the proportionally large uptake of NPs by the reticuloendothelial system (RES), preventing NPs from reaching desired sites in sufficient concentration. Alone, intraarterial (IA) delivery of NPs directly into the blood supply of the desired area of local therapy suffers from a dwell time that is not optimal to allow for effective uptake of NPs by desired tissues.

Thus, in some aspects a composition is provided comprising a polynucleotide-functionalized nanoparticle and an embolic agent. In various aspects, the polynucleotide is RNA, DNA or a modified polynucleotide. In one aspect, the polynucleotide is an antagomiR.

In further aspects, the polynucleotide is double stranded or in some aspects the polynucleotide is single stranded. In some aspects where the polynucleotide is double stranded, one strand of the double stranded polynucleotide is a guide strand. In some aspects, the polynucleotide comprises a detectable marker.

In various embodiments, the embolic agent is selected from the group consisting of a lipid emulsion (for example and without limitation, ethiodized oil or lipiodol), gelatin sponge, tris acetyl gelatin microspheres, embolization coils, ethanol, small molecule drugs, biodegradable microspheres, non-biodegradable microspheres or polymers, and self-assemblying embolic material.

In some embodiments, the functionalized nanoparticle and the embolic agent are present in a ratio of about 1:1 to about 10:1. In some embodiments, the functionalized nanoparticle and the embolic agent are present in a ratio of about 2:1 to about 5:1. In further embodiments, the functionalized nanoparticle and the embolic agent are present in a ratio of about 3:1.

In alternative aspects of the disclosure, the functionalized nanoparticle and the embolic agent are present in a ratio of about 1:2 to about 1:10. In related aspects, the functionalized nanoparticle and the embolic agent are present in a ratio of 1:3 to about 1:6. In further aspects, the functionalized nanoparticle and the embolic agent are present in a ratio of about 1:4.

In some aspects of the disclosure that pertain to a ratio, the ratio is a molar ratio. In other aspects, the ratio is volume to volume. In further aspects, the ratio is the number of nanoparticles to the number of embolic agent molecules.

In various aspects, a composition of the disclosure further comprises a therapeutic agent. In some embodiments, the therapeutic agent is associated with the nanoparticle.

In some embodiments, the therapeutic agent is selected from the group consisting of a protein, a chemotherapeutic agent, a radioactive material, a small molecule, and a polynucleotide.

The present disclosure additionally provides a method of local delivery of a composition disclosed herein comprising the step of identifying the site for delivery and delivering the composition. In some aspects, the delivering step is to a site of pathogenesis. In some aspects, the identifying step is performed by interventional radiology.

In some aspects, the delivering step is performed intraarterially while in some aspects the delivering step is performed intravenously.

In some embodiments, the methods disclosed herein further comprise the step of administering an additional embolic agent, wherein the additional embolic agent is part of the composition. In alternative embodiments, the additional embolic agent is administered separately from the composition.

In some aspects, the additional embolic agent is administered before the composition. In further aspects, the additional embolic agent is administered after the composition.

In some embodiments of the methods, the pathogenesis is associated with a cancer. In various aspects, the cancer is selected from the group consisting of liver, pancreatic, stomach, colorectal, prostate, testicular, renal cell, breast, bladder, ureteral, brain, lung, connective tissue, hematological, cardiovascular, lymphatic, skin, bone, eye, nasopharyngeal, laryngeal, esophagus, oral membrane, tongue, thyroid, parotid, mediastinum, ovary, uterus, adnexal, small bowel, appendix, carcinoid, gall bladder, pituitary, cancer arising from metastatic spread, and cancer arising from endodermal, mesodermal or ectodermally-derived tissues.

In some embodiments, the pathogenesis is associated with a solid organ disease. In various aspects, the solid organ is selected from the group consisting of heart, liver, pancreas, prostate, brain, eye, thyroid, pituitary, parotid, skin, spleen, stomach, esophagus, gall bladder, small bowel, bile duct, appendix, colon, rectum, breast, bladder, kidney, ureter, lung, and a endodermally-, ectodermally- or mesodermally-derived tissue.

The present disclosure also provides methods, in some embodiments, wherein the delivery of the composition regulates the expression of a target polynucleotide. In various aspects of these embodiments, the target polynucleotide is survivin. In some aspects, the target polynucleotide is a microRNA (miRNA), and in further aspects the miRNA is miRNA 210. In further aspects, the target polynucleotide is KRAS, and in still further aspects, the target polynucleotide is p53.

In some embodiments, the delivering step is to a site of a solid organ. In various aspects, the solid organ is selected from the group consisting of heart, liver, pancreas, prostate, brain, eye, thyroid, pituitary, parotid, skin, spleen, stomach, esophagus, gall bladder, small bowel, bile duct, appendix, colon, rectum, breast, bladder, kidney, ureter, lung, and a endodermally-, ectodermally- or mesodermally-derived tissue.

In further embodiments, the identifying step is performed by interventional radiology. In further aspects, the delivering step is performed intraarterially while in some aspects the delivering step is performed intravenously.

In some aspects of the present disclosure, the delivery of the composition regulates the expression of a target polynucleotide.

The present disclosure also contemplates, in some embodiments, a second delivery of the composition. In various aspects, the second delivery of the composition is administered after 24 hours. In further aspects, subsequent administrations of the composition occur about daily, about weekly, about every other week, about monthly, about every 6 weeks, or about every other month. In still further aspects, the second delivery of the composition occurs within about a minute, about an hour, more than one day, about a week, or about a month following an initial administration of the composition.

Further aspects of the invention will become apparent from the detailed description provided below. However, it should be understood that the following detailed description and examples, while indicating preferred embodiments of the invention, are given by way of illustration only since various changes and modifications within the spirit and scope of the invention will become apparent to those skilled in the art from this detailed description.

### DESCRIPTION OF THE DRAWINGS

FIG. 1 depicts a scheme illustrating intraarterial drug delivery in a VX2 rabbit model of liver cancer. Dotted arrow represents direction of catheter-based drug delivery. Curved arrows represent reflux, and nontargeted drug delivery.

FIG. 2 depicts (A) Angiogram depicting vascular anatomy. LHA=Left hepatic artery, RHA=Right hepatic artery, Cath=Catheter. Dashed inset region magnified (B) demonstrating venous phase angiogram with hypervascular ‘tumor blush’ (arrows).

FIG. 3 depicts the biodistribution of gold nanoparticles (ng/g tissue) across various organs by delivery method.

### DETAILED DESCRIPTION OF THE INVENTION

Nanoparticles have emerged as an especially versatile platform for delivering therapeutics in vitro [Paciotti et al., Drug Deliv. 11 (3): 169-83 (2004); Dhar et al., J Am Chem Soc. 131 (41):14652-3 (2009); Gibson et al., J Am Chem Soc. 129 (37): 11653-61(2007)] and in vivo [Patra et al., Cancer Res. 68 (6): 1970-8 (2008)]. As reported by Mirkin et al. [Giljohann et al., Journal of the American Chemical Society. 131 (6): 2072-3 (2009); Seferos et al., Chembiochem. 8 (11): 1230-2 (2007); Prigodich et al., ACS Nano. 2009; 3 (8):2147-52 (2009); Rosi et al., Science. 312 (5776): 1027-30 (2006)], DNA functionalized gold nanoparticles (DNA-AuNPs) can regulate intracellular gene expression as a single agent transfection entity, with high cellular uptake and resistance to enzymatic degradation. Despite these promising results in cell culture, several studies in animal models have shown that systemic intravenous administration of gold nanoparticles results in rapid sequestration by organs of the reticuloendothelial system (normal liver and spleen) for long durations, regardless of size, shape, and dose [Balasubramanian et al., Biomaterials. 31 (8): 2034-42 (2010); Sadauskas et al., Nanomedicine : nanotechnology, biology, and medicine. 5 (2): 162-9 (2009)]. Thus, traditional intravenous administration may limit the concentration of nanotherapeutics in target cells, while leading to unnecessary accumulation in normal liver tissue. Local delivery of nanoparticles has the potential to enhance therapeutic efficacy and reduce these off-target effects.

Embolic agents increase localized drug concentration, while decreasing drug washout by decreasing arterial inflow. Agents of this type have been shown to be preferentially retained in target cells [Kan et al., Invest Radiol. 29 (11): 990-3 (1994); Ohishi Radiology. 154(1): 25-9 (1985)], while being rapidly cleared by healthy tissue [Kan et al., Invest Radiol. 29 (11): 990-3 (1994); Kan et al., Radiology. 186 (3): 861-6 (1993); Okayasu et al., Am J Clin Pathol. 90 (5):536-44 (1988)]. Thus, drug concentrations can be increased within target cells [Cha et al., Curr Probl Surg. 47 (1): 10-67 (2010)] enhancing the desired therapeutic effect.

Nanoparticle-based therapeutics represent a novel means to overcome the limitations of current treatment modalities through either drug delivery or intracellular gene regulation [Ghosh et al., Adv Drug Deliv Rev. 60 (11): 1307-15 (2008)]. Furthermore, nanoparticle platforms minimize degradation and maximize solubility of their payload, while delivering high concentration of therapeutics to target tissues [Ozpolat et al., J Intern Med. 267 (1): 44-53 (2010)].

Accordingly, in some embodiments the present disclosure provides a composition comprising a polynucleotide-functionalized nanoparticle and an embolic agent. Throughout the disclosure, the term “functionalized” is used interchangeably with the terms “attached” and “bound.”

### Nanoparticles

Compositions of the present disclosure comprise nanoparticles as described herein. Nanoparticles are provided which are functionalized to have a polynucleotide attached thereto. The size, shape and chemical composition of the nanoparticles contribute to the properties of the resulting PN-NP. These properties include for example, optical properties, optoelectronic properties, electrochemical properties, electronic properties, stability in various solutions, magnetic properties, and pore and channel size variation. Mixtures of nanoparticles having different sizes, shapes and/or chemical compositions, as well as the use of nanoparticles having uniform sizes, shapes and chemical composition, and therefore a mixture of properties are contemplated. Examples of suitable particles include, without limitation, aggregate particles, isotropic (such as spherical particles), anisotropic particles (such as non-spherical rods, tetrahedral, and/or prisms) and core-shell particles, such as those described in U.S. Pat. No. 7,238,472 and International Publication No. WO 2003/08539, the disclosures of which are incorporated by reference in their entirety.

In one embodiment, the nanoparticle is metallic, and in various aspects, the nanoparticle is a colloidal metal. Thus, in various embodiments, nanoparticles of the invention include metal (including for example and without limitation, silver, gold, platinum, aluminum, palladium, copper, cobalt, indium, nickel, or any other metal amenable to nanoparticle formation), semiconductor (including for example and without limitation, CdSe, CdS, and CdS or CdSe coated with ZnS) and magnetic (for example, ferromagnetite) colloidal materials.

Also, as described in U.S. Patent Publication No 2003/0147966, nanoparticles of the invention include those that are available commercially, as well as those that are synthesized, e.g., produced from progressive nucleation in solution (e.g., by colloid reaction) or by various physical and chemical vapor deposition processes, such as sputter deposition. See, e.g., HaVashi, Vac. Sci. Technol. A5 (4) :1375-84 (1987); Hayashi, Physics Today, 44-60 (1987); MRS Bulletin, Jan. 1990, 16-47. As further described in U.S. Patent Publication No 2003/0147966, nanoparticles contemplated are alternatively produced using HAuCl₄ and a citrate-reducing agent, using methods known in the art. See, e.g., Marinakos et al., Adv. Mater. 11:34-37 (1999); Marinakos et al., Chem. Mater. 10: 1214-19 (1998); Enustun & Turkevich, J. Am. Chem. Soc. 85: 3317 (1963).

Nanoparticles can range in size from about 1 nm to about 250 nm in mean diameter, about 1 nm to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 nm in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, or about 1 nm to about 20 nm in mean diameter, about 1 nm to about 10 nm in mean diameter. In other aspects, the size of the nanoparticles is from about 5 nm to about 150 nm (mean diameter), from about 5 to about 50 nm, from about 10 to about 30 nm, from about 10 to 150 nm, from about 10 to about 100 nm, or about 10 to about 50 nm. The size of the nanoparticles is from about 5 nm to about 150 nm (mean diameter), from about 30 to about 100 nm, from about 40 to about 80 nm. The size of the nanoparticles used in a method varies as required by their particular use or application. The variation of size is advantageously used to optimize certain physical characteristics of the nanoparticles, for example, optical properties or the amount of surface area that can be functionalized as described herein.

### Polynucleotides

The terms “polynucleotide” and “nucleotide” or plural forms as used herein are interchangeable with modified forms as discussed herein and otherwise known in the art. In certain instances, the art uses the term “nucleobase” which embraces naturally-occurring nucleotides as well as modifications of nucleotides that can be polymerized. Thus, nucleotide or nucleobase means the naturally occurring nucleobases adenine (A), guanine (G), cytosine (C), thymine (T) and uracil (U) as well as non-naturally occurring nucleobases such as xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C₃-C₆)-alkynyl-cytosine, 5-fluorouracil, 5-bromouracil, pseudoisocytosine, 2-hydroxy-5-methyl-4-triazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, polynucleotides also include one or more “nucleosidic bases” or “base units” which include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Polynucleotides may also include modified nucleobases. A “modified base” is understood in the art to be one that can pair with a natural base (e.g., adenine, guanine, cytosine, uracil, and/or thymine) and/or can pair with a non-naturally occurring base. Exemplary modified bases are described in EP 1 072 679 and WO 97/12896, the disclosures of which are incorporated herein by reference. Modified nucleobases include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing the binding affinity and include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. No. 3,687,808, U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

Nanoparticles provided that are functionalized with a polynucleotide, or modified form thereof, generally comprise a polynucleotide from about 5 nucleotides to about 100 nucleotides in length. More specifically, nanoparticles are functionalized with polynucleotide that are about 5 to about 90 nucleotides in length, about 5 to about 80 nucleotides in length, about 5 to about 70 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, and all polynucleotides intermediate in length of the sizes specifically disclosed to the extent that the polynucleotide is able to achieve the desired result. Accordingly, polynucleotides of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides in length are contemplated.

In various aspects, the polynucleotide that is attached to the nanoparticle is single stranded. In some aspects, the polynucleotide that is attached to the nanoparticle is double stranded. In various aspects wherein the polynucleotide that is attached to the nanoparticle, one strand of the double stranded polynucleotide is a guide strand.

Guide strands are polynucleotide sequences designed to be complementary (antisense) to transcribed RNAs of any upregulated protein in, for example and without limitation, any human malignancy as determined by prior investigations (Scheme 1, dashed strands). Sequences that are complementary to these guide strands (Scheme 1 solid strands) are synthesized and attached to thiolated O-ethylene glycol (OEG) (Scheme 1, bolded solid strands) and loaded onto the NP surface. Guide strands are then duplexed to thiolated OEG strands to produce the final product (Scheme 1).

Polynucleotides contemplated for attachment to a nanoparticle include those which modulate expression of a gene product expressed from a target polynucleotide. The polynucleotides may, in various aspects, be comprised of DNA or RNA. Accordingly, antisense polynucleotides which hybridize to a target polynucleotide and inhibit translation, siRNA polynucleotides which hybridize to a target polynucleotide and initiate an RNAse activity (for example but not limited to RNAse H), triple helix forming polynucleotides which hybridize to double-stranded polynucleotides and inhibit transcription, and ribozymes which hybridize to a target polynucleotide and inhibit translation, are contemplated.

In some embodiments, the polynucleotide that is attached to the nanoparticle is an antagomiR. An antagomiR represents a novel class of chemically engineered polynucleotides. AntagomiRs are used to silence endogenous microRNA (miRNA) [Krützfeldt et al., Nature 438 (7068): 685-9 (2005)]. AntagomiRs are, in some aspects, covalently modified with lipophoilic groups (for example and without limitation, cholesterol), or other agents specifically used to image the location of the antagomiR (for example and without limitation, a molecular fluorophore).

In various aspects, if a specific mRNA is targeted, a single nanoparticle-binding agent composition has the ability to bind to multiple copies of the same transcript. In one aspect, a nanoparticle is provided that is functionalized with identical polynucleotides, i.e., each polynucleotide has the same length and the same sequence. In other aspects, the nanoparticle is functionalized with two or more polynucleotides which are not identical, i.e., at least one of the attached polynucleotides differ from at least one other attached polynucleotide in that it has a different length and/or a different sequence. In aspects wherein different polynucleotides are attached to the nanoparticle, these different polynucleotides bind to the same single target polynucleotide but at different locations, or substrate sites, or bind to different target polynucleotides which encode different gene products. Accordingly, in various aspects, a single nanoparticle-binding agent composition target more than one gene product. Polynucleotides are thus target-specific polynucleotides, whether at one or more specific regions in the target polynucleotide, or over the entire length of the target polynucleotide as the need may be to effect a desired level of inhibition of gene expression.

### Modified Polynucleotides

Modified polynucleotides are contemplated for functionalizing nanoparticles wherein both one or more sugar and/or one or more internucleotide linkage of the nucleotide units in the polynucleotide is replaced with “non-naturally occurring” groups. In one aspect, this embodiment contemplates a peptide nucleic acid (PNA). In PNA compounds, the sugar-backbone of a polynucleotide is replaced with an amide containing backbone. See, for example U.S. Pat. Nos. 5,539,082; 5,714,331; and 5,719,262, and Nielsen et al., Science, 1991, 254, 1497-1500, the disclosures of which are herein incorporated by reference.

Other linkages between nucleotides and unnatural nucleotides contemplated for the disclosed polynucleotides include those described in U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920; U.S. Patent Publication No. 20040219565; International Patent Publication Nos. WO 98/39352 and WO 99/14226; Mesmaeker et. al., Current Opinion in Structural Biology 5:343-355 (1995) and Susan M. Freier and Karl-Heinz Altmann, Nucleic Acids Research, 25:4429-4443 (1997), the disclosures of which are incorporated herein by reference.

Specific examples of polynucleotides include those containing modified backbones or non-natural internucleoside linkages. Polynucleotides having modified backbones include those that retain a phosphorus atom in the backbone and those that do not have a phosphorus atom in the backbone. Modified polynucleotides that do not have a phosphorus atom in their internucleoside backbone are considered to be within the meaning of “polynucleotide.”

Modified polynucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are polynucleotides having inverted polarity comprising a single 3′ to 3′ linkage at the 3′-most internucleotide linkage, i.e. a single inverted nucleoside residue which may be abasic (the nucleotide is missing or has a hydroxyl group in place thereof). Salts, mixed salts and free acid forms are also contemplated.

Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified polynucleotide backbones that do not include a phosphorus atom have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. In still other embodiments, polynucleotides are provided with phosphorothioate backbones and oligonucleosides with heteroatom backbones, and including —CH₂—NH—O—CH₂—, —CH₂—N(CH₃)—O—CH₂—, —CH₂—O—N(CH₃)—CH₂—, —CH₂—N(CH₃)—N(CH₃)—CH₂— and —O—N(CH₃)—CH₂—CH₂— described in U.S. Pat. Nos. 5,489,677, and 5,602,240. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

In various forms, the linkage between two successive monomers in the oligo consists of 2 to 4, desirably 3, groups/atoms selected from —CH₂, —O—, —S—, —NRH—, >C═O, >C═NRH, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHRH)—, where RH is selected from hydrogen and C1-4-alkyl, and R″ is selected from C1-6-alkyl and phenyl. Illustrative examples of such linkages are —CH₂—CH₂—CH₂—, —CH₂—CO—CH₂—, —CH₂—CHOH—CH₂—, O—CH2—O—, —O—CH2—CH2—, —O—CH2—CH═(including R5 when used as a linkage to a succeeding monomer), —CH₂—CH₂—O—, —NRH—CH₂—CH₂—, —CH₂—CH₂—NRH—, —CH₂—NRH—CH₂—, —O—CH₂—CH₂—NRH—, —NRH—CO—O—, —NRH—CO—NRH—, —NRH—CS—NRH—, —NRH—C(═NRH)—NRH—, —NRH—CO—CH₂—NRH—O—CO—O—, —O—CO—CH₂—O—, —O—CH₂—CO—O—, —CH₂—CO—NRH—, —O—CO—NRH—, —NRH—CO—CH₂—, —O—CH₂—CO—NRH—, —O——CH₂—CH₂—NRH—, —CH═N—O—, —CH₂—NRH—O—, —CH₂—O—N═(including R5 when used as a linkage to a succeeding monomer), —CH₂—O—NRH—, —CO—NRH—CH₂—, —CH₂—NRH—O—, —CH₂—NRH—CO—, —O—NRH—CH₂—, —O—NRH, —O—CH₂—S—, S—CH₂—O—, —CH₂—CH₂—S—, —O—CH₂—CH₂—S—, —S—CH₂—CH═(including R5 when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NRH—, —NRH—S(O)₂—CH₂—; —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—-P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(O CH₂CH₃)—O—, —O—PO(O CH₂CH₂S—R)—O—, —O—PO(BH₃)—O—, —O—PO(NHRN)—O—, —O—P(O)₂—NRH H—, —NRH—P(O)₂—O—, —O—P(O,NRH)—O—, —CH₂—P(O)₂—O—, —O—P(O)₂—CH₂—, and —O—Si(R″)₂—O—; among which —CH₂—CO—NRH—, —CH₂—NRH—O—, —S—CH₂—O—, —O—P(O)₂—O—O—P(—O,S)—O—, —O—P(S)₂—O—, NRH P(O)₂—O—, —O—P(O,NRH)—O—, —O—PO(R″)—O—, —O—PO(CH₃)—O—, and —O—PO(NHRN)—O—, where RH is selected form hydrogen and C1-4-alkyl, and R″ is selected from C1-6-alkyl and phenyl, are contemplated. Further illustrative examples are given in Mesmaeker et. al., 1995, Current Opinion in Structural Biology, 5: 343-355 and Susan M. Freier and Karl-Heinz Altmann, 1997, Nucleic Acids Research, vol 25: pp 4429-4443.

Still other modified forms of polynucleotides are described in detail in U.S. Patent Application No. 20040219565, the disclosure of which is incorporated by reference herein in its entirety.

Modified polynucleotides may also contain one or more substituted sugar moieties. In certain aspects, polynucleotides comprise one of the following at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Other embodiments include O[(CH₂)ₙO]ₘCH₃, O(CH2)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other polynucleotides comprise one of the following at the 2′ position: C1 to C10 lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a polynucleotide, or a group for improving the pharmacodynamic properties of a polynucleotide, and other substituents having similar properties. In one aspect, a modification includes 2′-methoxyethoxy (2′-O—CH₂CH₂OCH₃, also known as 2′-O—(2-methoxyethyl) or 2′-MOE) (Martin et al., 1995, Hely. Chim. Acta, 78: 486-504) i.e., an alkoxyalkoxy group. Other modifications include 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Still other modifications include 2′-methoxy (2′-O—CH₃), 2′-aminopropoxy (2′-OCH₂CH₂CH₂NH₂), 2′-allyl (2′-CH₂—CH=CH₂), 2′-O-allyl (2′-O—CH₂—CH═CH₂) and 2′-fluoro (2′-F). The 2′-modification may be in the arabino (up) position or ribo (down) position. In one aspect, a 2′-arabino modification is 2′-F. Similar modifications may also be made at other positions on the polynucleotide, for example, at the 3′ position of the sugar on the 3′ terminal nucleotide or in 2′-5′ linked polynucleotides and the 5′ position of 5′ terminal nucleotide. Polynucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In one aspect, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is in certain aspects a methylene (—CH₂—)n group bridging the 2′ oxygen atom and the 4′ carbon atom wherein n is 1 or 2. LNAs and preparation thereof are described in WO 98/39352 and WO 99/14226, the disclosures of which are incorporated herein by reference.

### Methods of Attaching Polynucleotides

Polynucleotides contemplated for use in the methods include those bound to the nanoparticle through any means. Regardless of the means by which the polynucleotide is attached to the nanoparticle, attachment in various aspects is effected through a 5′ linkage, a 3′ linkage, some type of internal linkage, or any combination of these attachments.

In one aspect, the nanoparticles, the polynucleotides or both are functionalized in order to attach the polynucleotides to the nanoparticles. Methods to functionalize nanoparticles and polynucleotides are known in the art. For instance, polynucleotides functionalized with alkanethiols at their 3′-termini or 5′-termini readily attach to gold nanoparticles. See Whitesides, Proceedings of the Robert A. Welch Foundation 39th Conference On Chemical Research Nanophase Chemistry, Houston, Tex., pages 109-121 (1995). See also, Mucic et al. [Chem. Commun. 555-557 (1996)] which describes a method of attaching 3′ thiol DNA to flat gold surfaces. The alkanethiol method can also be used to attach polynucleotides to other metal, semiconductor and magnetic colloids and to the other types of nanoparticles described herein. Other functional groups for attaching polynucleotides to solid surfaces include phosphorothioate groups (see, for example, U.S. Pat. No. 5,472,881 for the binding of polynucleotide-phosphorothioates to gold surfaces), substituted alkylsiloxanes [(see, for example, Burwell, Chemical Technology, 4, 370-377 (1974) and Matteucci and Caruthers, J. Am. Chem. Soc., 103, 3185-3191 (1981) for binding of polynucleotides to silica and glass surfaces, and Grabar et al., [Anal. Chem., 67, 735-743] for binding of aminoalkylsiloxanes and for similar binding of mercaptoaklylsiloxanes]. Polynucleotides with a 5′ thionucleoside or a 3′ thionucleoside may also be used for attaching polynucleotides to solid surfaces. The following references describe other methods which may be employed to attached polynucleotides to nanoparticles: Nuzzo et al., J. Am. Chem. Soc., 109, 2358 (1987) (disulfides on gold); Allara and Nuzzo, Langmuir, 1, 45 (1985) (carboxylic acids on aluminum); Allara and Tompkins, J. Colloid Interface Sci., 49, 410-421 (1974) (carboxylic acids on copper); Iler, The Chemistry Of Silica, Chapter 6, (Wiley 1979) (carboxylic acids on silica); Timmons and Zisman, J. Phys. Chem., 69, 984-990 (1965) (carboxylic acids on platinum); Soriaga and Hubbard, J. Am. Chem. Soc., 104, 3937 (1982) (aromatic ring compounds on platinum); Hubbard, Acc. Chem. Res., 13, 177 (1980) (sulfolanes, sulfoxides and other functionalized solvents on platinum); Hickman et al., J. Am. Chem. Soc., 111, 7271 (1989) (isonitriles on platinum); Maoz and Sagiv, Langmuir, 3, 1045 (1987) (silanes on silica); Maoz and Sagiv, Langmuir, 3, 1034 (1987) (silanes on silica); Wasserman et al., Langmuir, 5, 1074 (1989) (silanes on silica); Eltekova and Eltekov, Langmuir, 3, 951 (1987) (aromatic carboxylic acids, aldehydes, alcohols and methoxy groups on titanium dioxide and silica); Lec et al., J. Phys. Chem., 92, 2597 (1988) (rigid phosphates on metals).

U.S. patent application Ser. Nos. 09/760,500 and 09/820,279 and international application nos. PCT/US01/01190 and PCT/US01/10071 describe polynucleotides functionalized with a cyclic disulfide. The cyclic disulfides in certain aspects have 5 or 6 atoms in their rings, including the two sulfur atoms. Suitable cyclic disulfides are available commercially or are synthesized by known procedures. Functionalization with the reduced forms of the cyclic disulfides is also contemplated. Functionalization with triple cyclic disulfide anchoring groups are described in PCT/US2008/63441, incorporated herein by reference in its entirety.

In certain aspects wherein cyclic disulfide functionalization is utilized, polynucleotides are attached to a nanoparticle through one or more linkers. In one embodiment, the linker comprises a hydrocarbon moiety attached to a cyclic disulfide. Suitable hydrocarbons are available commercially, and are attached to the cyclic disulfides. The hydrocarbon moiety is, in one aspect, a steroid residue. Polynucleotide-nanoparticle compositions prepared using linkers comprising a steroid residue attached to a cyclic disulfide are more stable compared to compositions prepared using alkanethiols or acyclic disulfides as the linker, and in certain instances, the polynucleotide-nanoparticle compositions have been found to be 300 times more stable. In certain embodiments the two sulfur atoms of the cyclic disulfide are close enough together so that both of the sulfur atoms attach simultaneously to the nanoparticle. In other aspects, the two sulfur atoms are adjacent each other. In aspects where utilized, the hydrocarbon moiety is large enough to present a hydrophobic surface screening the surfaces of the nanoparticle.

In other aspects, a method for attaching polynucleotides onto a surface is based on an aging process described in U.S. application Ser. No. 09/344,667, filed Jun. 25, 1999; Ser. No. 09/603,830, filed Jun. 26, 2000; Ser. No. 09/760,500, filed Jan. 12, 2001; Ser. No. 09/820,279, filed Mar. 28, 2001; Ser. No. 09/927,777, filed Aug. 10, 2001; and in International application nos. PCT/US97/12783, filed Jul. 21, 1997; PCT/US00/17507, filed Jun. 26, 2000; PCT/US01/01190, filed Jan. 12, 2001; PCT/US01/10071, filed Mar. 28, 2001, the disclosures which are incorporated by reference in their entirety. The aging process provides nanoparticle-polynucleotide compositions with enhanced stability and selectivity. The process comprises providing polynucleotides, in one aspect, having covalently bound thereto a moiety comprising a functional group which can bind to the nanoparticles. The moieties and functional groups are those that allow for binding (i.e., by chemisorption or covalent bonding) of the polynucleotides to nanoparticles. For example, polynucleotides having an alkanethiol, an alkanedisulfide or a cyclic disulfide covalently bound to their 5′ or 3′ ends bind the polynucleotides to a variety of nanoparticles, including gold nanoparticles.

Compositions produced by use of the “aging” step have been found to be considerably more stable than those produced without the “aging” step. Increased density of the polynucleotides on the surfaces of the nanoparticles is achieved by the “aging” step. The surface density achieved by the “aging” step will depend on the size and type of nanoparticles and on the length, sequence and concentration of the polynucleotides. A surface density adequate to make the nanoparticles stable and the conditions necessary to obtain it for a desired combination of nanoparticles and polynucleotides can be determined empirically. Generally, a surface density of at least 2 picomoles/cm² will be adequate to provide stable nanoparticle-polynucleotide compositions. Regardless, various polynucleotide densities are contemplated as disclosed herein.

An “aging” step is incorporated into production of functionalized nanoparticles following an initial binding or polynucleotides to a nanoparticle. In brief, the polynucleotides are contacted with the nanoparticles in water for a time sufficient to allow at least some of the polynucleotides to bind to the nanoparticles by means of the functional groups. Such times can be determined empirically. In one aspect, a time of about 12-24 hours is contemplated. Other suitable conditions for binding of the polynucleotides can also be determined empirically. For example, a concentration of about 10-20 nM nanoparticles and incubation at room temperature is contemplated.

Next, at least one salt is added to the water to form a salt solution. The salt is any water-soluble salt, including, for example and without limitation, sodium chloride, magnesium chloride, potassium chloride, ammonium chloride, sodium acetate, ammonium acetate, a combination of two or more of these salts, or one of these salts in phosphate buffer. The salt is added as a concentrated solution, or in the alternative as a solid. In various embodiments, the salt is added all at one time or the salt is added gradually over time. By “gradually over time” is meant that the salt is added in at least two portions at intervals spaced apart by a period of time. Suitable time intervals can be determined empirically.

The ionic strength of the salt solution must be sufficient to overcome at least partially the electrostatic repulsion of the polynucleotides from each other and, either the electrostatic attraction of the negatively-charged polynucleotides for positively-charged nanoparticles, or the electrostatic repulsion of the negatively-charged polynucleotides from negatively-charged nanoparticles. Gradually reducing the electrostatic attraction and repulsion by adding the salt gradually over time gives the highest surface density of polynucleotides on the nanoparticles. Suitable ionic strengths can be determined empirically for each salt or combination of salts. In one aspect, a final concentration of sodium chloride of from about 0.01 M to about 1.0 M in phosphate buffer is utilized , with the concentration of sodium chloride being increased gradually over time. In another aspect, a final concentration of sodium chloride of from about 0.01 M to about 0.5 M, or about 0.1 M to about 0.3 M is utilized, with the concentration of sodium chloride being increased gradually over time.

After adding the salt, the polynucleotides and nanoparticles are incubated in the salt solution for a period of time to allow additional polynucleotides to bind to the nanoparticles to produce the stable nanoparticle-polynucleotide compositions. An increased surface density of the polynucleotides on the nanoparticles stabilizes the compositions, as has been described herein. The time of this incubation can be determined empirically. By way of example, in one aspect a total incubation time of about 24-48, wherein the salt concentration is increased gradually over this total time, is contemplated. This second period of incubation in the salt solution is referred to herein as the “aging” step. Other suitable conditions for this “aging” step can also be determined empirically. By way of example, an aging step is carried out with incubation at room temperature and pH 7.0.

The compositions produced by use of the “aging” are in general more stable than those produced without the “aging” step. As noted above, this increased stability is due to the increased density of the polynucleotides on the surfaces of the nanoparticles which is achieved by the “aging” step. The surface density achieved by the “aging” step will depend on the size and type of nanoparticles and on the length, sequence and concentration of the polynucleotides.

As used herein, “stable” means that, for a period of at least six months after the compositions are made, a majority of the polynucleotides remain attached to the nanoparticles and the polynucleotides are able to hybridize with nucleic acid and polynucleotide targets under standard conditions encountered in methods of detecting nucleic acid and methods of nanofabrication.

### Surface Density

Nanoparticles as provided herein have a packing density of the polynucleotides on the surface of the nanoparticle that is, in various aspects, sufficient to result in cooperative behavior between nanoparticles and between polynucleotide strands on a single nanoparticle. In another aspect, the cooperative behavior between the nanoparticles increases the resistance of the polynucleotide to nuclease degradation. In yet another aspect, the uptake of nanoparticles by a cell is influenced by the density of polynucleotides associated with the nanoparticle. As described in PCT/US2008/65366, incorporated herein by reference in its entirety, a higher density of polynucleotides on the surface of a nanoparticle is associated with an increased uptake of nanoparticles by a cell.

A surface density adequate to make the nanoparticles stable and the conditions necessary to obtain it for a desired combination of nanoparticles and polynucleotides can be determined empirically. Generally, a surface density of at least 2 pmoles/cm² will be adequate to provide stable nanoparticle-polynucleotide compositions. In some aspects, the surface density is at least 15 pmoles/cm². Methods are also provided wherein the polynucleotide is bound to the nanoparticle at a surface density of at least 2 pmol/cm², at least 3 pmol/cm², at least 4 pmol/cm², at least 5 pmol/cm², at least 6 pmol/cm², at least 7 pmol/cm², at least 8 pmol/cm², at least 9 pmol/cm², at least 10 pmol/cm², at least about 15 pmol/cm², at least about 20 pmol/cm², at least about 25 pmol/cm², at least about 30 pmol/cm², at least about 35 pmol/cm², at least about 40 pmol/cm², at least about 45 pmol/cm², at least about 50 pmol/cm², at least about 55 pmol/cm², at least about 60 pmol/cm², at least about 65 pmol/cm², at least about 70 pmol/cm², at least about 75 pmol/cm², at least about 80 pmol/cm², at least about 85 pmol/cm², at least about 90 pmol/cm², at least about 95 pmol/cm², at least about 100 pmol/cm², at least about 125 pmol/cm², at least about 150 pmol/cm², at least about 175 pmol/cm², at least about 200 pmol/cm², at least about 250 pmol/cm², at least about 300 pmol/cm², at least about 350 pmol/cm², at least about 400 pmol/cm², at least about 450 pmol/cm², at least about 500 pmol/cm², at least about 550 pmol/cm², at least about 600 pmol/cm², at least about 650 pmol/cm², at least about 700 pmol/cm², at least about 750 pmol/cm², at least about 800 pmol/cm², at least about 850 pmol/cm², at least about 900 pmol/cm², at least about 950 pmol/cm², at least about 1000 pmol/cm² or more.

Density of polynucleotides on the surface of a nanoparticle has been shown to modulate specific polypeptide interactions with the polynucleotide on the surface and/or with the nanoparticle itself. Under various conditions, some polypeptides may be prohibited from interacting with polynucleotides associated with a nanoparticle based on steric hindrance caused by the density of polynucleotides. In aspects where interaction of polynucleotides with polypeptides that are otherwise precluded by steric hindrance is desirable, the density of polynucleotides on the nanoparticle surface is decreased to allow the polypeptide to interact with the polynucleotide.

Polynucleotide surface density has also been shown to modulate stability of the polynucleotide associated with the nanoparticle. In one embodiment, an RNA polynucleotide associated with a nanoparticle is provided wherein the RNA polynucleotide has a half-life that is at least substantially the same as the half-life of an identical RNA polynucleotide that is not associated with a nanoparticle. In other embodiments, the RNA polynucleotide associated with the nanoparticle has a half-life that is about 5% greater, about 10% greater, about 20% greater, about 30% greater, about 40% greater, about 50% greater, about 60% greater, about 70% greater, about 80% greater, about 90% greater, about 2-fold greater, about 3-fold greater, about 4-fold greater, about 5-fold greater, about 6-fold greater, about 7-fold greater, about 8-fold greater, about 9-fold greater, about 10-fold greater, about 20-fold greater, about 30-fold greater, about 40-fold greater, about 50-fold greater, about 60-fold greater, about 70-fold greater, about 80-fold greater, about 90-fold greater, about 100-fold greater, about 200-fold greater, about 300-fold greater, about 400-fold greater, about 500-fold greater, about 600-fold greater, about 700-fold greater, about 800-fold greater, about 900-fold greater, about 1000-fold greater, about 5000-fold greater, about 10,000-fold greater, about 50,000-fold greater, about 100,000-fold greater, about 200,000-fold greater, about 300,000-fold greater, about 400,000-fold greater, about 500,000-fold greater, about 600,000-fold greater, about 700,000-fold greater, about 800,000-fold greater, about 900,000-fold greater, about 1,000,000-fold greater or more than the half-life of an identical RNA polynucleotide that is not associated with a nanoparticle.

### Polynucleotide Features

The present disclosure provides, in various embodiments, PN-NP compositions that are useful for gene regulation. In some aspects, the PN-NP is functionalized with DNA. In some embodiments, the DNA is double stranded, and in further embodiments the DNA is single stranded. In further aspects, the PN-NP is functionalized with RNA, and in still further aspects the PN-NP is functionalized with double stranded RNA agents known as small interfering RNA (siRNA). The term “RNA” includes duplexes of two separate strands, as well as single stranded structures. Single stranded RNA also includes RNA with secondary structure. In one aspect, RNA having a hairpin loop in contemplated.

Polynucleotides that are contemplated for use in gene regulation and functionalized to a nanoparticle have complementarity to (i.e., are able to hybridize with) a portion of a target RNA (generally messenger RNA (mRNA)).

“Hybridization” means an interaction between two or three strands of nucleic acids by hydrogen bonds in accordance with the rules of Watson-Crick complementarity, Hoogstein binding, or other sequence-specific binding known in the art. Hybridization can be performed under different stringency conditions known in the art.

Generally, such complementarity is 100%, but can be less if desired, such as about 20%, about 25%, about 30%, about 35%, about 40%, about 45%, about 50%, about 55%, about 60%, about 70%, about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. For example, 19 bases out of 21 bases may be base-paired. Thus, it will be understood that a polynucleotide used in the methods need not be 100% complementary to a desired target nucleic acid to be specifically hybridizable. Moreover, polynucleotides may hybridize to each other over one or more segments such that intervening or adjacent segments are not involved in the hybridization event (e.g., a loop structure or hairpin structure). Percent complementarity between any given polynucleotide can be determined routinely using BLAST programs (Basic Local Alignment Search Tools) and PowerBLAST programs known in the art (Altschul et al., 1990, J. Mol. Biol., 215: 403-410; Zhang and Madden, 1997, Genome Res., 7: 649-656).

In some aspects, where selection between various allelic variants is desired, 100% complementarity to the target gene is required in order to effectively discern the target sequence from the other allelic sequence. When selecting between allelic targets, choice of length is also an important factor because it is the other factor involved in the percent complementary and the ability to differentiate between allelic differences.

### Target Polynucleotide Sequences And Hybridization

In some aspects, the disclosure provides methods of targeting specific polynucleotide. Any type of polynucleotide may be targeted, and the methods may be used, e.g., for therapeutic modulation of gene expression (See, e.g., PCT/US2006/022325, the disclosure of which is incorporated herein by reference). Examples of polynucleotides that can be targeted by the methods of the invention include but are not limited to genes (e.g., a gene associated with a particular disease), viral RNA, mRNA, RNA, or single-stranded nucleic acids.

The target nucleic acid may be in cells, tissue samples, or biological fluids, as also known in the art. See, e.g., Sambrook et al., Molecular Cloning: A Laboratory Manual (2nd ed. 1989) and B. D. Hames and S. J. Higgins, Eds., Gene Probes 1 (IRL Press, New York, 1995).

The terms “start codon region” and “translation initiation codon region” refer to a portion of a mRNA or gene that encompasses contiguous nucleotides in either direction (i.e., 5′ or 3′) from a translation initiation codon. Similarly, the terms “stop codon region” and “translation termination codon region” refer to a portion of such a mRNA or gene that encompasses contiguous nucleotides in either direction (i.e., 5′ or 3′) from a translation termination codon. Consequently, the “start codon region” (or “translation initiation codon region”) and the “stop codon region” (or “translation termination codon region”) are all regions which may be targeted effectively with the polynucleotides on the functionalized nanoparticles.

Other target regions include the 5′ untranslated region (5′UTR), the portion of an mRNA in the 5′ direction from the translation initiation codon, including nucleotides between the 5′ cap site and the translation initiation codon of a mRNA (or corresponding nucleotides on the gene), and the 3′ untranslated region (3′UTR), the portion of a mRNA in the 3′ direction from the translation termination codon, including nucleotides between the translation termination codon and 3′ end of a mRNA (or corresponding nucleotides on the gene). The 5′ cap site of a mRNA comprises an N7-methylated guanosine residue joined to the 5′-most residue of the mRNA via a 5′-5′ triphosphate linkage. The 5′ cap region of a mRNA is considered to include the 5′ cap structure itself as well as the first 50 nucleotides adjacent to the cap site.

For prokaryotic target nucleic acid, in various aspects, the nucleic acid is RNA transcribed from genomic DNA. For eukaryotic target nucleic acid, the nucleic acid is an animal nucleic acid, a plant nucleic acid, a fungal nucleic acid, including yeast nucleic acid. As above, the target nucleic acid is a RNA transcribed from a genomic DNA sequence. In certain aspects, the target nucleic acid is a mitochondrial nucleic acid. For viral target nucleic acid, the nucleic acid is viral genomic RNA, or RNA transcribed from viral genomic DNA.

In some embodiments of the disclosure, a target polynucleotide sequence is a microRNA. MicroRNAs (miRNAs) are 20-22 nucleotide (nt) molecules generated from longer 70-nt RNAs that include an imperfectly complementary hairpin segment [Jackson et al., Sci STKE 367: rel (2007); Mendell, Cell Cycle 4: 1179-1184 (2005)]. The longer precursor molecules are cleaved by a group of proteins (Drosha and DCGR8) in the nucleus into smaller RNAs called pre-miRNA. Pre-miRNAs are then exported into the cytoplasm by exportin [Virmani et al., J Vasc Intery Radiol 19: 931-936 (2008)] proteins. The pre-miRNA in the cytoplasm is then cleaved into mature RNA by a complex of proteins called RNAi silencing complex or RISC. The resulting molecule has 19-bp double stranded RNA and 2 nt 3′ overhangs on both strands. One of the two strands is then expelled from the complex and is degraded. The resulting single strand RNA-protein complex can then inhibit translation (either by repressing the actively translating ribosomes or by inhibiting initiation of translation) or enhance degradation of the mRNA it is attached to. There is, of course, a high degree of selectivity to this process, as the miRNA only binds to areas that are of high match to its sequence [Zamore et al., Science 309: 1519-1524 (2005)]. In one aspect, the target polynucleotide is microRNA-210.

Methods for inhibiting gene product expression provided include those wherein expression of the target gene product is inhibited by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% compared to gene product expression in the absence of an polynucleotide-functionalized nanoparticle. In other words, methods provided embrace those which results in essentially any degree of inhibition of expression of a target gene product.

The degree of inhibition is determined in vivo from a body fluid sample or from a biopsy sample or by imaging techniques well known in the art. Alternatively, the degree of inhibition is determined in a cell culture assay, generally as a predictable measure of a degree of inhibition that can be expected in vivo resulting from use of a specific type of nanoparticle and a specific polynucleotide.

### Embolic Agents

The present disclosure employs the use of a polynucleotide-functionalized nanoparticle in combination with an embolic agent. As discussed above, embolic agents serve to increase localized drug concentration in target sites through selective occlusion of blood vessels by purposely introducing emboli, while decreasing drug washout by decreasing arterial inflow. In various aspects of the compositions and methods of the disclosure, the embolic agent is selected from the group consisting of a lipid emulsion (for example and without limitation, ethiodized oil or lipiodol), gelatin sponge, tris acetyl gelatin microspheres, embolization coils, ethanol, small molecule drugs, biodegradable microspheres, non-biodegradable microspheres or polymers, and self-assemblying embolic material.

The present disclosure describes compositions and methods to deliver nanoparticles locally to a site of pathogenesis. This local delivery is termed “nanoembolization.” In various embodiments, PN-NP particles are mixed with the embolic agent just prior to administration. The PN-NP/embolic agent mixture may be used alone for nanoembolization, or may be followed by administration of another embolic agent microspheres.

It has been shown that intraarterial (IA) delivery alone does now allow for dwell time at the desired site of therapy that is sufficient for efficient uptake of therapeutic PN-NPs. Thus the addition of embolic agent allows the therapy to block blood flow to a desired site increasing the dwell time of injected therapeutics which keeps the local concentration of therapeutic high and enhances delivery to tissue. Thus, using IA delivery of nanoparticles (NP) combined with an embolic agent greatly increases NP concentration in the vicinity of target cells and limits their distribution throughout the rest of the body, thereby greatly improving NP uptake in targeted cells of interest.

Compositions of the present disclosure comprise ratios of PN-NPs and embolic agent. “Ratio,” as used herein, can be a molar ratio, a volume to volume ratio or it can be the number of PN-NPs to the number of embolic agent molecules. One of ordinary skill in the art can determine the ratio to be used in the compositions of the present disclosure.

In some embodiments, the PN-NPs and the embolic agent are present in a ratio of about 1:1 to about 10:1. In further embodiments, the PN-NPs and the embolic agent are present in a ratio of about 2:1 to about 5:1. In one aspect, the PN-NPs and the embolic agent are present in a ratio of about 3:1. The present disclosure contemplates, in various aspects, that compositions of PN-NPs and the embolic agent are present in a ratio of about 1:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 31:1, about 32:1, about 33:1, about 34:1, about 35:1, about 36:1, about 37:1, about 38:1, about 39:1, about 40:1, about 41:1, about 42:1, about 43:1, about 44:1, about 45:1, about 46:1, about 47:1, about 48:1, about 49:1, about 50:1, about 55:1, about 60:1, about 65:1, about 70:1, about 75:1, about 80:1, about 85:1, about 90:1, about 95:1, about 100:1, about 150:1, about 200:1, about 250:1, about 300:1, about 350:1, about 400:1, about 450:1, about 500:1, about 550:1, about 600:1, about 650:1, about 700:1, about 750:1, about 800:1, about 850:1, about 900:1, about 950:1, about 1000:1, about 2000:1, about 5000:1, about 7000:1, about 10000:1 or greater.

In alternative aspects, compositions of PN-NPs and the embolic agent are present in a ratio of about 1:2, about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, about 1:20, about 1:21, about 1:22, about 1:23, about 1:24, about 1:25, about 1:26, about 1:27, about 1:28, about 1:29, about 1:30, about 1:31, about 1:32, about 1:33, about 1:34, about 1:35, about 1:36, about 1:37, about 1:38, about 1:39, about 1:40, about 1:41, about 1:42, about 1:43, about 1:44, about 1:45, about 1:46, about 1:47, about 1:48, about 1:49, about 1:50, about 1:55, about 1:60, about 1:65, about 1:70, about 1:75, about 1:80, about 1:85, about 1:90, about 1:95, about 1:100, about 1:150, about 1:200, about 1:250, about 1:300, about 1:350, about 1:400, about 1:450, about 1:500, about 1:550, about 1:600, about 1:700, about 1:750, about 1:800, about 1:850, about 1:900, about 1:950, about 1:1000, about 1:2000, about 1:5000, about 1:10000 or greater.

In further embodiments, the PN-NPs are approximately lnanomolar (nM) to 10 micromolar (μM), while the embolic agent is in the μM to millimolar (mM) range. Accordingly, in some embodiments, this would yield PN-NP:embolic agent ratios of about 1:1, about 1:10, about 1:100, about 1:1000, about 1:10,000 or higher.

### Target Site Identification and Composition Delivery

Methods provided include those wherein a composition of the disclosure is locally delivered to a target site. Once the target site has been identified, a composition of the disclosure is delivered, in one aspect, intraarterially. In another aspect, a composition of the disclosure is delivered intravenously.

Target site identification is performed, in some aspects, by interventional radiology. For example and without limitation, an IR procedure is performed in which a catheter is advanced into the artery directly supplying a tumor to be treated under image guidance. Perfusion of the tumor is confirmed, then the PN-NP/embolic agent composition is injected, with or without injection of an additional embolic agent. In aspects where an additional embolic agent is administered, the additional embolic agent can be part of the composition or, in some aspects, can be administered separately from the composition. In aspects where the additional embolic agent is administered separately from the composition, it is contemplated that the additional embolic agent can be administered before or after the composition.

Intraarterial drug delivery, pioneered by the field of interventional radiology (IR), has been used extensively in the minimally invasive treatment of a wide variety of diseases including solid tumors. IR physicians are able to catheterize the blood supply directly feeding a solid tumor and deliver relatively high doses of chemotherapeutics while limiting the systemic side effects of such drugs. This process is followed by the administration of an embolic agent to block blood flow to the tumor starving it of nutrients and increasing the dwell time of injected therapeutics, keeping the local concentration of chemotherapeutic high. Using IA delivery of gold nanoparticles, either in conjunction with an embolic agent or followed by injection of an embolic agent, greatly increases NP concentration in tumor cells and limits their distribution throughout the rest of the body, thus greatly improving their uptake in cancer cells.

For nanoembolization, a vascular catheter is advanced superselectively under fluoroscopic guidance into a tumor's feeding artery. Therapeutic nanoparticles are then infused through the catheter, along with embolic agents, with the goal of maximizing intratumoral drug concentration. This material is used, for example and without limitation, for the treatment of cancer as described above, the delivery of therapeutic agents for tissue regeneration or growth of tissue, or for the delivery of molecularly targeted imaging agents.

Image-Guided Nanoembolization takes advantage of a number of imaging modalities including MRI, CT, X-Ray DSA or ultrasound to guide catheter placement, confirm tumor perfusion, and deliver NPs locally.

In various aspects, the target site is a site of pathogenesis.

In some aspects, the site of pathogenesis is cancer. In various aspects, the cancer is selected from the group consisting of liver, pancreatic, stomach, colorectal, prostate, testicular, renal cell, breast, bladder, ureteral, brain, lung, connective tissue, hematological, cardiovascular, lymphatic, skin, bone, eye, nasopharyngeal, laryngeal, esophagus, oral membrane, tongue, thyroid, parotid, mediastinum, ovary, uterus, adnexal, small bowel, appendix, carcinoid, gall bladder, pituitary, cancer arising from metastatic spread, and cancer arising from endodermal, mesodermal or ectodermally-derived tissues.

In some embodiments, the site of pathogenesis is a solid organ disease. In various aspects, the solid organ is selected from the group consisting of heart, liver, pancreas, prostate, brain, eye, thyroid, pituitary, parotid, skin, spleen, stomach, esophagus, gall bladder, small bowel, bile duct, appendix, colon, rectum, breast, bladder, kidney, ureter, lung, and a endodermally-, ectodermally- or mesodermally-derived tissues.

In some embodiments, a second delivery of a composition as described herein is performed. In various aspects, the second delivery of the composition is administered after 24 hours. In further aspects, subsequent administrations of the composition occur about daily, about weekly, about every other week, about monthly, about every 6 weeks, or about every other month. In still further aspects, the second delivery of the composition occurs within about a minute, about an hour, more than one day, about a week, or about a month following an initial administration of the composition.

In some embodiments, the second delivery of the composition occurs within about 2 minutes, about 3 minutes, about 4 minutes, about 5 minutes, about 6 minutes, about 7 minutes, about 8 minutes, about 9 minutes, about 10 minutes, about 15 minutes, about 20 minutes, about 30 minutes, about 40 minutes, about 50 minutes, about 60 minutes, about 8 hours, about 2 days, about 3 days, about 4 days, about 5 days, about 6 days, about 10 days, about 15 days, about 20 days, about 25 days or more following an initial administration of the composition.

These schedules, in various aspects, would follow the chemotherapy paradigm of treating patients with a series of doses, separated in time to optimize therapeutic benefit, while minimizing toxicity. Each single dosing would, in various aspects, take minutes to hours to deliver. In some aspects, an administration schedule comprises continuous intraarterial administration using an implantable catheter that occurs, in various aspects, over a time course of days to weeks.

It is also contemplated by the present disclosure that the compositions disclosed herein are useful for diagnostic purposes. In some embodiments, administration of a composition of the disclosure is used to detect the presence of an aberrant polynucleotide that is indicative of a disease in a biological sample. Methods of detecting a polynucleotide using a functionalized nanoparticle are generally described in International Application No. PCT/US2008/053603, the entire disclosure of which is incorporated by reference herein in its entirety.

### Detectable Marker

Methods are provided wherein presence of a polynucleotide is detected by an observable change. In one aspect, presence of the polynucleotide gives rise to a color change which is observed with a device capable of detecting a specific marker as disclosed herein. For example and without limitation, a fluorescence microscope can detect the presence of a fluorophore that is conjugated to a polynucleotide, which has been functionalized on a nanoparticle.

It will be understood that a marker contemplated will include any of the fluorophores described herein as well as other detectable markers known in the art. For example, markers also include, but are not limited to, redox active probes, other nanoparticles, and quantum dots, as well as any marker which can be detected using spectroscopic means, i.e., those markers detectable using microscopy and cytometry. In various aspects, isotopes are contemplated as a general method of identifying the location of embolized material. In further aspects, imaging contrast agents (for example and without limitation, gadolinium and/or fluorine) are contemplated as a general method of identifying the location of embolized material.

Suitable fluorescent molecules are also well known in the art and include without limitation 1,8-ANS (1-Anilinonaphthalene-8-sulfonic acid), 1-Anilinonaphthalene-8-sulfonic acid (1,8-ANS), 5-(and-6)-Carboxy-2′, 7′-dichlorofluorescein pH 9.0, 5-FAM pH 9.0, 5-ROX (5-Carboxy-X-rhodamine, triethylammonium salt), 5-ROX pH 7.0, 5-TAMRA, 5-TAMRA pH 7.0, 5-TAMRA-MeOH, 6 JOE, 6,8-Difluoro-7-hydroxy-4-methylcoumarin pH 9.0, 6-Carboxyrhodamine 6G pH 7.0, 6-Carboxyrhodamine 6G, hydrochloride, 6-HEX, SE pH 9.0, 6-TET, SE pH 9.0, 7-Amino-4-methylcoumarin pH 7.0, 7-Hydroxy-4-methylcoumarin, 7-Hydroxy-4-methylcoumarin pH 9.0, Alexa 350, Alexa 405, Alexa 430, Alexa 488, Alexa 532, Alexa 546, Alexa 555, Alexa 568, Alexa 594, Alexa 647, Alexa 660, Alexa 680, Alexa 700, Alexa Fluor 430 antibody conjugate pH 7.2, Alexa Fluor 488 antibody conjugate pH 8.0, Alexa Fluor 488 hydrazide-water, Alexa Fluor 532 antibody conjugate pH 7.2, Alexa Fluor 555 antibody conjugate pH 7.2, Alexa Fluor 568 antibody conjugate pH 7.2, Alexa Fluor 610 R-phycoerythrin streptavidin pH 7.2, Alexa Fluor 647 antibody conjugate pH 7.2, Alexa Fluor 647 R-phycoerythrin streptavidin pH 7.2, Alexa Fluor 660 antibody conjugate pH 7.2, Alexa Fluor 680 antibody conjugate pH 7.2, Alexa Fluor 700 antibody conjugate pH 7.2, Allophycocyanin pH 7.5, AMCA conjugate, Amino Coumarin, APC (allophycocyanin) ,Atto 647, BCECF pH 5.5, BCECF pH 9.0, BFP (Blue Fluorescent Protein), BO-PRO-1-DNA, BO-PRO-3-DNA, BOBO-1-DNA, BOBO-3-DNA, BODIPY 650/665-X, MeOH, BODIPY FL conjugate, BODIPY FL, MeOH, Bodipy R6G SE, BODIPY R6G, MeOH, BODIPY TMR-X antibody conjugate pH 7.2, Bodipy TMR-X conjugate, BODIPY TMR-X, MeOH, BODIPY TMR-X, SE, BODIPY TR-X phallacidin pH 7.0, BODIPY TR-X, MeOH, BODIPY TR-X, SE, BOPRO-1, BOPRO-3, Calcein, Calcein pH 9.0, Calcium Crimson, Calcium Crimson Ca2+, Calcium Green, Calcium Green-1 Ca2+, Calcium Orange, Calcium Orange Ca2+, Carboxynaphthofluorescein pH 10.0, Cascade Blue, Cascade Blue BSA pH 7.0, Cascade Yellow, Cascade Yellow antibody conjugate pH 8.0, CFDA, CFP (Cyan Fluorescent Protein), CI-NERF pH 2.5, CI-NERF pH 6.0, Citrine, Coumarin, Cy 2, Cy 3, Cy 3.5, Cy 5, Cy 5.5, CyQUANT GR-DNA, Dansyl Cadaverine, Dansyl Cadaverine, MeOH, DAPI, DAPI-DNA, Dapoxyl (2-aminoethyl) sulfonamide, DDAO pH 9.0, Di-8 ANEPPS, Di-8-ANEPPS-lipid, DiI, DiO, DM-NERF pH 4.0, DM-NERF pH 7.0, DsRed, DTAF, dTomato, eCFP (Enhanced Cyan Fluorescent Protein), eGFP (Enhanced Green Fluorescent Protein), Eosin, Eosin antibody conjugate pH 8.0, Erythrosin-5-isothiocyanate pH 9.0, Ethidium Bromide, Ethidium homodimer, Ethidium homodimer-1-DNA, eYFP (Enhanced Yellow Fluorescent Protein), FDA, FITC, FITC antibody conjugate pH 8.0, FlAsH, Fluo-3, Fluo-3 Ca2+, Fluo-4, Fluor-Ruby, Fluorescein, Fluorescein 0.1 M NaOH, Fluorescein antibody conjugate pH 8.0, Fluorescein dextran pH 8.0, Fluorescein pH 9.0, Fluoro-Emerald, FM 1-43, FM 1-43 lipid, FM 4-64, FM 4-64, 2% CHAPS, Fura Red Ca2+, Fura Red, high Ca, Fura Red, low Ca, Fura-2 Ca2+, Fura-2, high Ca, Fura-2, no Ca, GFP (S65T), HcRed, Hoechst 33258, Hoechst 33258-DNA, Hoechst 33342, Indo-1 Ca2+, Indo-1, Ca free, Indo-1, Ca saturated, JC-1, JC-1 pH 8.2, Lissamine rhodamine, LOLO-1-DNA, Lucifer Yellow, CH, LysoSensor Blue, LysoSensor Blue pH 5.0, LysoSensor Green, LysoSensor Green pH 5.0, LysoSensor Yellow pH 3.0, LysoSensor Yellow pH 9.0, LysoTracker Blue, LysoTracker Green, LysoTracker Red, Magnesium Green, Magnesium Green Mg2+, Magnesium Orange, Marina Blue, mBanana, mCherry, mHoneydew, MitoTracker Green, MitoTracker Green FM, MeOH, MitoTracker Orange, MitoTracker Orange, MeOH, MitoTracker Red, MitoTracker Red, MeOH, mOrange, mPlum, mRFP, mStrawberry, mTangerine, NBD-X, NBD-X, MeOH, NeuroTrace 500/525, green fluorescent Nissl stain-RNA, Nile Blue, EtOH, Nile Red, Nile Red-lipid, Nissl, Oregon Green 488, Oregon Green 488 antibody conjugate pH 8.0, Oregon Green 514, Oregon Green 514 antibody conjugate pH 8.0, Pacific Blue, Pacific Blue antibody conjugate pH 8.0, Phycoerythrin, PicoGreen dsDNA quantitation reagent, PO-PRO-1, PO-PRO-1-DNA, PO-PRO-3, PO-PRO-3-DNA, POPO-1, POPO-1-DNA, POPO-3, Propidium Iodide, Propidium Iodide-DNA, R-Phycoerythrin pH 7.5, ReAsH, Resorufin, Resorufin pH 9.0, Rhod-2, Rhod-2 Ca2+, Rhodamine, Rhodamine 110, Rhodamine 110 pH 7.0, Rhodamine 123, MeOH, Rhodamine Green, Rhodamine phalloidin pH 7.0, Rhodamine Red-X antibody conjugate pH 8.0, Rhodaminen Green pH 7.0, Rhodol Green antibody conjugate pH 8.0, Sapphire, SBFI-Na+, Sodium Green Na+, Sulforhodamine 101, EtOH, SYBR Green I, SYPRO Ruby, SYTO 13-DNA, SYTO 45-DNA, SYTOX Blue-DNA, Tetramethylrhodamine antibody conjugate pH 8.0, Tetramethylrhodamine dextran pH 7.0, Texas Red-X antibody conjugate pH 7.2, TO-PRO-1-DNA, TO-PRO-3-DNA, TOTO-1-DNA, TOTO-3-DNA, TRITC, X-Rhod-1 Ca2+, YO-PRO-1-DNA, YO-PRO-3-DNA, YOYO-1-DNA, and YOYO-3-DNA.

In yet another embodiment, two types of fluorescent-labeled polynucleotides attached to two different particles can be used. This may be useful, for example and without limitation, to track two different cell populations. Suitable particles include polymeric particles (such as, without limitation, polystyrene particles, polyvinyl particles, acrylate and methacrylate particles), glass particles, latex particles, Sepharose beads and others like particles well known in the art. Methods of attaching polynucleotides to such particles are well known and routinely practiced in the art. See Chrisey et al., 1996, Nucleic Acids Research, 24: 3031-3039 (glass) and Charreyre et al., 1997 Langmuir, 13: 3103-3110, Fahy et al., 1993, Nucleic Acids Research, 21: 1819-1826, Elaissari et al., 1998, J. Colloid Interface Sci., 202: 251-260, Kolarova et al., 1996, Biotechniques, 20: 196-198 and Wolf et al., 1987, Nucleic Acids Research, 15: 2911-2926 (polymer/latex).

Other labels besides fluorescent molecules can be used, such as chemiluminescent molecules, which will give a detectable signal or a change in detectable signal upon hybridization.

Methods of labeling polynucleotides with fluorescent molecules and measuring fluorescence are well known in the art.

### Therapeutic Agents

In some embodiments, a composition of the present disclosure further comprises a therapeutic agent. In some aspects, the therapeutic agent is associated with the nanoparticle. In other aspects, the therapeutic agent is co-administered with the PN-NP, but is separate from the PN-NP composition. In further aspects, the therapeutic agent is administered before the administration of the PN-NP composition, and in still further aspects, the therapeutic agent is administered after the administration of the PN-NP composition. One of ordinary skill in the art will understand that multiple therapeutic agents in multiple combinations can be administered at any time before, during or after administration of the PN-NP composition. In addition, repeated administration of a therapeutic agent is also contemplated.

In an embodiment of the invention, the therapeutic agent is selected from the group consisting of a protein, peptide, a chemotherapeutic agent, a small molecule, a radioactive material, and a polynucleotide.

Protein therapeutic agents include, without limitation peptides, enzymes, structural proteins, receptors and other cellular or circulating proteins as well as fragments and derivatives thereof, the aberrant expression of which gives rise to one or more disorders. Therapeutic agents also include, as one specific embodiment, chemotherapeutic agents. Still other therapeutic agents include polynucleotides, including without limitation, protein coding polynucleotides, polynucleotides encoding regulatory polynucleotides, and/or polynucleotides which are regulatory in themselves. Therapeutic agents also include, in various embodiments, a radioactive material.

In various aspects, protein therapeutic agents include cytokines or hematopoietic factors including without limitation IL-1 alpha, IL-1 beta, IL-2, IL-3, IL-4, IL-5, IL-6, IL-11, colony stimulating factor-1 (CSF-1), M-CSF, SCF, GM-CSF, granulocyte colony stimulating factor (G-CSF), EPO, interferon-alpha (IFN-alpha), consensus interferon, IFN-beta, IFN-gamma, IL-7, IL-8, IL-9, IL-10, IL-12, IL-13, IL-14, IL-15, IL-16, IL-17, IL-18, thrombopoietin (TPO), angiopoietins, for example Ang-1, Ang-2, Ang-4, Ang-Y, the human angiopoietin-like polypeptide, vascular endothelial growth factor (VEGF), angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor, cytokine-induced neutrophil chemotactic factor 1, cytokine-induced neutrophil, chemotactic factor 2α, cytokine-induced neutrophil chemotactic factor 2β, β endothelial cell growth factor, endothelin 1, epidermal growth factor, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor acidic, fibroblast growth factor basic, glial cell line-derived neutrophic factor receptor α1, glial cell line-derived neutrophic factor receptor α2, growth related protein, growth related protein a, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neurotrophin-3, neurotrophin-4, placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor a, platelet derived growth factor receptor β, pre-B cell growth stimulating factor, stem cell factor receptor, TNF, including TNF0, TNF1, TNF2, transforming growth factor a, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein III, tumor necrosis factor receptor type I, tumor necrosis factor receptor type II, urokinase-type plasminogen activator receptor, vascular endothelial growth factor, and chimeric proteins and biologically or immunologically active fragments thereof.

In other aspects, chemotherapeutic agent include, without limitation, alkylating agents including: nitrogen mustards, such as mechlor-ethamine, cyclophosphamide, ifosfamide, melphalan and chlorambucil; nitrosoureas, such as carmustine (BCNU), lomustine (CCNU), and semustine (methyl-CCNU); ethylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil, fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; epipodophylotoxins such as etoposide and teniposide; antibiotics such as actimomycin D, daunomycin (rubidomycin), doxorubicin, mitoxantrone, idarubicin, bleomycins, plicamycin (mithramycin), mitomycinC, and actinomycin; enzymes such as L-asparaginase; biological response modifiers such as interferon-alpha, IL-2, G-CSF and GM-CSF; miscellaneous agents including platinium coordination complexes such as cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide.

The term “small molecule,” as used herein, refers to a chemical compound, for instance a peptidometic or polynucleotide that may optionally be derivatized, or any other low molecular weight organic compound, either natural or synthetic. Such small molecules may be a therapeutically deliverable substance or may be further derivatized to facilitate delivery.

By “low molecular weight” is meant compounds having a molecular weight of less than 1000 Daltons, typically between 300 and 700 Daltons. Low molecular weight compounds, in various aspects, are about 100, about 150, about 200, about 250, about 300, about 350, about 400, about 450, about 500, about 550, about 600, about 650, about 700, about 750, about 800, about 850, about 900, about 1000 or more Daltons.

Polynucleotide therapeutic agents include, in one aspect and without limitation, those which encode therapeutic proteins described herein and otherwise known in the art, as well as polynucleotides which have intrinsic regulatory functions. Polynucleotides that have regulatory functions have been described herein above and include without limitation RNAi , antisense, ribozymes, and triplex-forming polynucleotides, each of which have the ability to regulate gene expression. Methods for carrying out these regulatory functions have previously been described in the art (Dykxhoom D M, Novina C D and Sharp P A, Nature Review, 4: 457-467, 2003; Mittal V, Nature Reviews, 5: 355-365, 2004).

It will be appreciated that, in various aspects, a therapeutic agent as described herein is attached to the nanoparticle.

### EXAMPLES

### Example 1

The aim of this study was to use the rabbit VX2 liver tumor model to show that nanoembolization increases PN-NP uptake in tumors over a) conventional intravenous systemic delivery and b) intra-arterial delivery without use of an embolic agent. An additional aim was to show that this approach minimizes off-target distribution of PN-NPs. Such results would be potentially applicable to other nanoparticle platforms and to any solid organ cancer that can be accessed locally via catheter. While the concept of delivering therapies by catheter is accepted clinically, the potential benefits of using a catheter to deliver therapeutic nanoparticles locally remains to be shown.

### Polynucleotide Synthesis

Citrate stabilized gold nanoparticles (13 nm diameter) were synthesized according to previously published protocols [Giljohann et al., Journal of the American Chemical Society. 131 (6): 2072-3 (2009); Seferos et al., Chembiochem. 8 (11): 1230-2 (2007); Prigodich et al., ACS Nano. 3 (8): 2147-52 (2009); Rosi et al., Science. 312 (5776): 1027-30.25-28 (2006)]. Polynucleotides were synthesized using an Expedite 8909 Nucleotide Synthesis System (Applied Biosystems, Foster City, Calif., USA) using standard solid-phase phosphoramidite methodology. All bases and reagents were purchased from Glen Research (Sterling, Va., USA). Following synthesis, polynucleotides were purified by reverse-phase high performance liquid chromatography (HPLC).

The polynucleotide sequence chosen for this experiment was an antagomiR to miR-210, known to be upregulated in HCC [Gramantieri et al., J Cell Mol Med. 12 (6A): 2189-204 (2008)], and involved in cancer cell survival under hypoxic conditions [Mathew et al., Mol Cell. 35 (6): 737-8 (2009); Huang et al., Mol Cell. 35 (6): 856-67 (2009)]. The sequence is as follows: 5′-CAG CCG TGT CAC ACG CAC AG-(A)10-propylthiol-3′ (SEQ ID NO: 1).

### Polynucleotide Gold Nanoparticle Conjugates

Alkyl-thiol-modified polynucleotides (final concentration 1 μM) were added to a 10 nM solution of 13±1 nm gold nanoparticles. After overnight incubation, sodium dodecylsulphate (SDS), phosphate buffer (pH=7.4), and sodium chloride were added to achieve final concentrations of 0.1%, 10 mM, and 0.1 M, respectively. An additional aliquot of sodium chloride was added to achieve a final concentration of 0.3 M, and the mixture was shaken overnight. The functionalized nanoparticles were purified from unreacted materials by three successive rounds of centrifugation (16 000 rcf, 20 min), supernatant removal, and resuspension in phosphate buffered saline (PBS) (137 mM NaCl, 10 mM phosphate, 2.7 mM KCl, pH 7.4, Hyclone, Thermo Scientific, Waltham, Mass., USA).

The concentrations of the purified nanoconjugates were determined by UV/vis spectrophotometry (λₘₐₓ=524 nm, ε=2.7×10⁸ mol⁻¹ cm⁻¹). Final injected concentration of nanoparticles injected in all animals was 100 nM.

### Animal Model

To examine intra-arterial nanoparticle delivery in the setting of HCC, we used the VX2 tumor rabbit model of HCC. This animal model was employed because similar to human HCC, the tumor blood supply is almost entirely derived from the hepatic artery [Ramirez et al., Invest New Drugs. 13 (1): 51-3 (1995)]. Tumors are also characterized by rapid growth [Kuszyk et al., Radiology 217 (2): 477-86 (2000)] and can be detected by multiple imaging modalities [Geschwind et al., J Vasc Intery Radiol. 11 (10): 1245-55 (2000)]. Additionally, rabbit hepatic arteries are of sufficient caliber to permit catheterization and direct infusion of therapeutic materials [Geschwind et al., J Vasc Intery Radiol. 11 (10): 1245-55 (2000)]. All animal studies were approved by our Institutional Animal Care and Use Committee (IACUC).

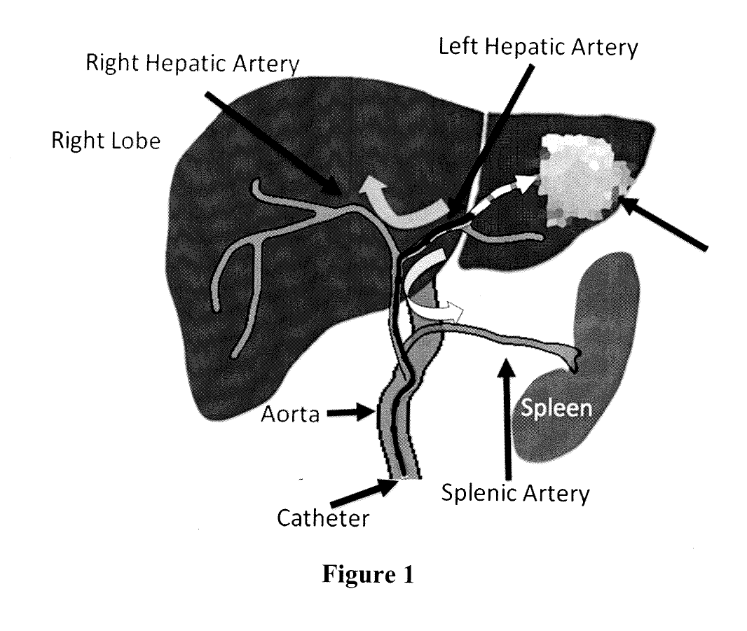

All experiments were performed using 9 New Zealand white rabbits weighing approximately 4-5 kg. VX2 cells were initially grown in the hindlimb of an additional donor rabbit (three-week incubation period). Hindlimb tumors were harvested, and small tumor sections (2-3 mm) were dissected from viable tumor tissue. VX2 tumors were surgically implanted in the left lateral lobe of the liver in 9 New Zealand white rabbits, as previously described (FIG. 1) [Virmani et al., Journal of vascular and interventional radiology : JVIR. 19 (6): 931-6 (2008)]. The right lobe of the liver was used as a tumor-free control. For surgical VX2 tumor liver implantation, rabbits were anesthetized with intramuscular (IM) ketamine 44 mg/kg and xylazine 3-5 mg/kg and supplemental inhalational isoflurane (2-3%) as needed.

After the animal was prepped and draped in the typical sterile fashion, a subxiphoid mini-laparotomy was performed, exposing the left lobe of the liver. Using a number 11 blade, a 1-2 cm incision was made across the liver capsule. One tumor section (2-3 mm) was placed within the incision. Hemostasis was achieved with gentle pressure and by placing a lx1 cm piece of surgicel (Ethicon, Somerville, NJ, USA) over the incision site, as previously described [Virmani et al., Journal of vascular and interventional radiology : JVIR. 19(6): 931-6 (2008)]. The abdomen was then closed in 3 layers. Liver tumors were incubated for approximately 3 weeks prior to imaging, to permit adequate growth.

### MR Imaging

MR imaging was performed with a 1.5-T clinical unit (Magnetom Espree; Siemens Medical Solutions, Erlangen, Germany). Rabbits were imaged in the supine position with use of a flexible surface coil and were intubated using a 3-F endotracheal tube with inhalational isoflurane (2-3.5%) anesthesia provided using a small-animal ventilator (Harvard Apparatus, Holliston, Mass., USA). Three weeks after tumor implantation, each rabbit underwent MR imaging to detect tumor growth. Tumor growth was considered positive when tumor was identified in axial and sagittal imaging planes by two independent MR imaging specialists.

Anatomic images of the liver tumors in all 9 rabbits were obtained by using a T2-weighted turbo spin-echo sequence with the following imaging parameters: 5,020/84 (repetition time msec/echo time msec), 5-mm-thick sections, 205-Hz per pixel bandwidth, 200×112-mm² field of view, 192×108 matrix, turbo factor 11, and four signals acquired.

### Treatment Groups

Following confirmation of tumor growth, animals were randomized to one of three treatment groups based on nanoparticle administration route: a) Intravenous (IV; n=3); b) Intra-arterial (IA; n=3); and c) nanoembolization (n=3), which was comprised of IA delivery of DNA-AuNPs emulsified in lipiodol, an iodinated oily embolic agent (ethiodized oil, Ethiodol; Savage Laboratories, Melville, N.Y.). Lipiodol offered several concurrent benefits. As an emulsifier, its avidity for tumors is an excellent delivery vehicle for the DNA-AuNPs. As a microvessel embolic agent, it reduces blood flow to the tumor and thus washout of the injected DNA-AuNPs. As an imaging contrast agent, it is radio-opaque and can thus be used to identify the nanoparticle emulsion under X-ray guidance in real-time during delivery. This radio-opacity also obviates the need to employ complex methods [Song et al., Angew Chem Int Ed Engl. 48 (48): 9143-7 (2009)] to attach imaging contrast agents to the nanoparticles.

All animals were initially sedated with a mixture of IM ketamine (80 mg/kg) and xylazine (5 mg/kg). For all treatment groups, animals received 3 mL of 100 nM DNA-AuNPs over a 5-minute infusion period. In the IV delivery group, animals were administered DNA-AuNPs through an ear vein cannula.

### X-Ray DSA

For IA delivery, the left hepatic artery supplying the tumor was accessed using a catheter advanced superselectively from the femoral artery under X-ray digital subtraction angiography (DSA) guidance (FIG. 1). X-ray DSA was performed using a Siemens C-arm PowerMobil unit (Siemens Medical Solutions, Erlangen, Germany). The 6 animals undergoing intra-arterial delivery were initially sedated with a mixture of IM ketamine (80 mg/kg) and xylazine (5 mg/kg). The animals were subsequently intubated.

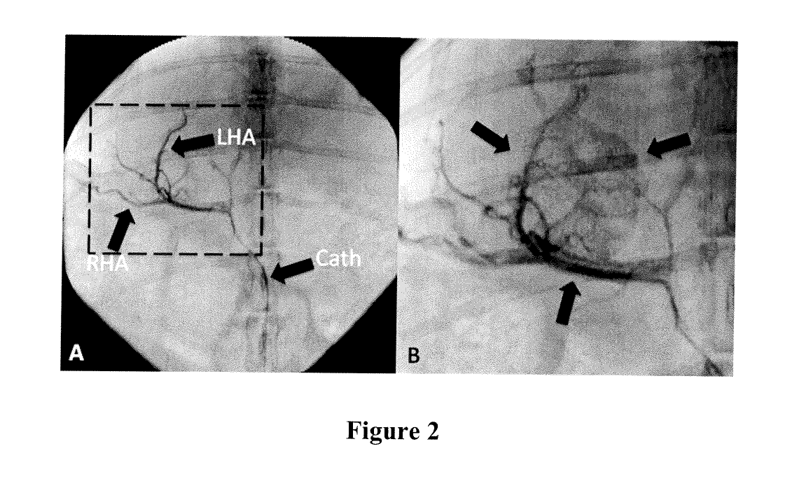

Using a surgical cutdown, the common femoral artery was isolated and catheterized using a 3-F vascular sheath (Cook, Bloomington, IN, USA). A 2-F catheter (Cook JB-1) was then advanced superselectively over a 0.014-inch diameter guidewire into the left hepatic artery that supplied the targeted tumor. Prior to injection of nanoparticles, DSA of the left hepatic artery was performed using 2 mL manual injections of an iodinated contrast agent (Omnipaque 350, Amersham Health, Princeton, N.J., USA) to delineate vascular anatomy (FIG. 2).

Following DSA confirmation of catheter position, the catheter was secured in place using a 2-0 silk suture in the rabbits' groin.

Under fluoroscopic guidance, rabbits received a 3 mL of 100 nM functionalized gold nanoparticles. For the nanoembolization group, a 1:3 solution was created by mixing lipiodol (ethiodized oil, Ethiodol; Savage Laboratories, Melville, N.Y.) with the nanoparticle solution. Of note, nanoparticle delivery could be detected by fluoroscopy only in the nanoembolization group, due the radio-opacity of the lipiodol nanoparticle emulsion. Delivery in the IA group without embolization was not visible fluoroscopically.

Tissue Harvest

Rabbits were kept alive for 4 hours after injection. This time point was selected based upon the desire to balance animal sedation and comfort, with sufficient time allotted for nanoparticles to enter cells. Animals were euthanized with Beuthanasia (100 mg/kg; Schering-Plough, Union, N.J., USA) 4 hours following nanoparticle administration. Tissue was harvested from organs (Table 1, below) known to harbor high concentrations of nanoparticles following systemic administration, according to previous reports [Balasubramanian et al., Biomaterials 31 (8): 2034-42 (2010)]. Tissue was obtained from the tumor (periphery and core), organs of the reticuloendothelial system (liver and spleen), kidneys and lungs. Each organ was divided into four quadrants, and a specimen was taken from each quadrant. Samples were placed in sterile eppendorf tubes for inductively coupled mass spectrometry analysis (ICP-MS). Samples were frozen in liquid nitrogen and stored at −80° C. until analysis.

### ICP-MS

Gold content within the tissue (nanograms/grams tissue) was determined by ICP-MS. Tissue samples were weighed using a dual range balance (Mettler Toledo XS105, Columbus, Ohio, USA), and then dissolved in 500 μL of trace metal grade nitric acid and incubated at 55° C. for 18 hours. To control for variation in preparation and instrument sampling, 400 μL of each sample was added to 3.6 mL of matrix buffer containing 2% HCl, 2% HNO₃ and 5 ppb Iridium internal standard. Gold concentrations of each sample were measured using ICP-Q-MS (VG PG Excel, Thermo Elemental inductively coupled plasma mass spectrometer and a PC running PlasmaLab software, Thermo Scientific, Waltham, Mass., USA). Gold content levels were averaged according to organ of origin. Differences between gold levels between treatment groups were compared with one-way ANOVA with Bonferroni's multiple comparison post-hoc tests with p<0.05 considered significant.

### Results

Following IV administration of nanoparticles, considerable uptake was noted in the non-tumor portions of the liver (right lobe: 273892±70263 ng/left lobe: 236422±56440 ng/g) and spleen (830918±207597 ng/g). These two sites comprise the major organs of the reticuloendothelial system. There was significantly less uptake of nanoparticles within the tumor itself (periphery: 59713±43501 ng/g, core: 6414±4865 ng/g; p<0.05). With IA delivery, a similar distribution was noted with no statistically significant difference compared to IV delivery in the liver or within the tumor itself. Of note, significantly higher nanoparticle uptake was measured in the spleen with IA versus IV delivery (1495558±137545 ng/g vs. 830918±207597 ng/g; p<0.05). Both IV and IA delivery resulted in nanoparticle uptake in the lungs and kidneys (Table 1), however this was significantly less than that measured in either the liver or spleen (p<0.05).

TABLE 1
Organ ICP-MS analysis of gold content by delivery method

| | Gold Content: ng/g tissue | | |
|---|---|---|---|
| Organ | Nanoembolization | Intra-arterial | Intravenous |
| Spleen | 246426 ± 67497 | 1495558 ± 137545 | 830918 ± 207597 |
| Kidneys | 5426 ± 3302 | 5489 ± 3873 | 4465 ± 3842 |
| Lungs | 21365 ± 13329 | 25020 ± 11051 | 20129 ± 18475 |
| Right Lobe | 176692 ± 107950 | 246830 ± 124676 | 273892 ± 70263 |
| Liver | | | |
| Left Lobe | 327210 ± 46868 | 246411 ± 118453 | 236422 ± 56440 |
| Liver | | | |
| Tumor | 590502 ± 80877 | 44904 ± 28858 | 59713 ± 43501 |
| Periphery | | | |
| Tumor Core | 97668 ± 23658 | 11233 ± 7371 | 6414 ± 4865 |

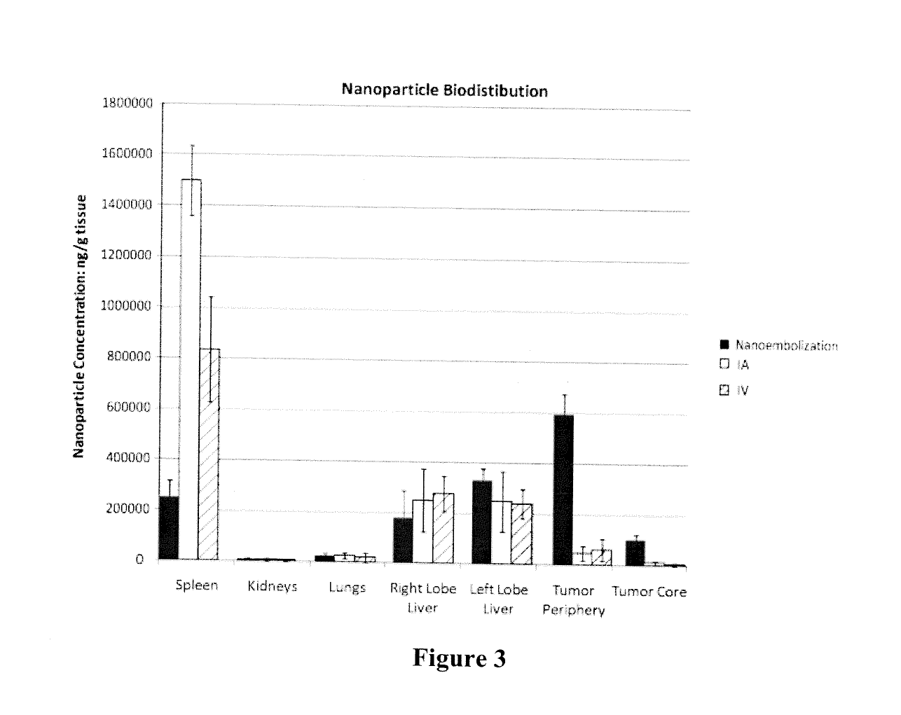

As depicted in FIG. 3, nanoembolization produced significantly higher concentration of nanoparticles within the tumor compared to IA or IV delivery (periphery: 590502±80877 ng/g, core: 97668±23658 ng/g; p<0.05). With this technique, more nanoparticles were delivered to the tumor than surrounding liver tissue (tumor periphery vs. right or left lobe of liver; p<0.05), which was not observed in the other two treatment groups. Nanoembolization significantly increased nanoparticle uptake in both the tumor periphery (10 and 13 times higher than IV and IA respectively; p<0.05) and tumor core (9 and 15 times higher than IV and IA respectively; p<0.05). There was also significantly less off-target delivery of nanoparticles to the spleen with nanoembolization versus IV or IA delivery (p<0.05). Although nanoembolization delivered the highest amount of nanoparticles to the tumor core, this amount was still significantly less than that delivered to the tumor periphery or healthy liver tissue. This finding can be attributed to the necrosis of the central tumor core seen on pathologic exam and the concomitant reduced central tumor blood flow.

### Discussion

At presentation, the majority of patients with HCC are not candidates for curative therapy. This may be secondary to concomitant cirrhosis with limited hepatic reserve, locally advanced or multifocal disease, unfavorable anatomy, or medical co-morbidities [Lau et al., Ann Surg. 249 (1): 20-5 (2009)]. Furthermore, these patients with impaired hepatic function can tolerate limited systemic chemotherapy, resulting in inadequate drug delivery to their tumor burden. Thus, the targeted delivery of therapeutics directly to tumors maximizing bioavailability while minimizing systemic effects is desirable.

In this setting, loco-regional therapy has emerged as a promising treatment modality. Tumor directed therapies, such as chemoembolization have improved patient survival, but remain palliative, indicating room for improvement [Llovet et al., Lancet. 359 (9319): 1734-9 (2002); Lo et al., Hepatology. 35 (5): 1164-71 (2002); A comparison of lipiodol chemoembolization and conservative treatment for unresectable hepatocellular carcinoma. Groupe d'Etude et de Traitement du Carcinome Hepatocellulaire. N Engl J Med. 332 (19): 1256-61 (1995); Pelletier et al., J Hepatol. 29 (1): 129-34 (1998); Bruix et al., Hepatology 27 (6): 1578-83]8, 14-17 (1998)].

Gold nanoparticles are an emerging class of agents that can overcome the limitations of conventional therapeutics. Characterized by low inherent toxicity, relatively high surface area, and tunable size and stability, these agents have be utilized in a variety of therapeutic applications from chemotherapeutic drug delivery to intracellular gene regulation [Ghosh et al., Adv Drug Deliv Rev. 60 (11): 1307-15 (2008)]. These nanoconjugates have enhanced anticancer effects over their constituent therapeutic entities. This is due to improved pharmacokinetics and increased intratumoral and intracellular penetration due to the EPR effect [Davis et al., Nat Rev Drug Discov. 7 (9): 771-82 (2008)]. However, when administered systemically, gold nanoparticles are overwhelmingly sequestered by organs of the RES, limiting their uptake in target tissues [Balasubramanian et al., Biomaterials 31 (8): 2034-42 (2010)]. This finding was confirmed by IV administration experiments. Systemic delivery therefore suffers from the same limitations as conventional IV therapeutics: 1) poor drug bioavailability; 2) nonspecific systemic distribution; and 3) inadequate intratumoral drug concentration [Gindy et al., Expert Opin Drug Deliv. 6 (8): 865-78 (2009)]. These shortcomings of IV administration hamper many of the benefits of nanoparticle based therapeutics. We therefore proposed adopting the transarterial approach, proven beneficial with conventional chemotherapeutics, to nanotherapeutics. Transarterial drug delivery has several proven advantages: 1) local administration increases local concentration; 2) the hemodynamics of the vascular bed can be altered with vasoactive agents (vasodilation or embolization); and 3) the prolonged dwell time of therapeutic agents results in greater efficacy [Arepally, J Magn Reson Imaging. 27 (2): 292-8 (2009)]. These principles of transarterial drug delivery have been applied to nanoparticle therapeutics to develop nanoembolization as described herein.

The benefits of nanoembolization to alter biodistribution compared to IV or IA administration are related to a synergistic effect of two factors: a) local delivery and b) embolization using lipiodiol. Local delivery alone showed no improvement in intratumoral nanoparticle uptake when comparing IA delivery to IV infusion. To dramatically increase intra-tumoral uptake in the liver, embolization was also required. Because embolic agents limit arterial inflow, they reduce nanoparticle washout, and increase dwell time allowing improved intratumoral nanoparticle penetration. Additionally, the radio-opacity of the selected embolic agent, lipiodol, enabled visualization of the injected nanoembolic emulsion during delivery, and thus helped avoid reflux into non-target organs. Because conventional DNA-AuNPs were not visible using x-ray imaging, it was not possible at the time of injection with IA delivery alone to verify whether the solution of nanoparticles went into the tumor, or refluxed into arteries supplying adjacent organs. This accounts for the high degree of splenic uptake noted with IA delivery. In distinction, because the DNA-AuNP/lipiodol emulsion could be readily seen during injection, reflux could be directly avoided, thereby leading to increased intra-tumoral uptake. Furthermore the use of lipiodol avoids the potential complexity and toxicity issues of attaching a contrast agent directly to the DNA-AuNPs.

Image-guided nanoembolization—the local catheter-based delivery of nanoparticles to the blood supply of tumor, followed by embolization—has been demonstrated herein to overcome two fundamental barriers to in vivo delivery of therapeutic nanoparticles: a) the poor uptake of nanoparticles into tumors and b) excessive non-target uptake in organs of the reticuloendothelial system with IV delivery. In addition to altering biodistribution, nanoembolization offers several other advantages. First, by using a radio-opaque, tumor-avid emulsion agent, the technique enables real-time visualization of nanoparticle delivery. Second, while this technique was investigated using DNA-AuNPs as the nanoconstruct, nanoembolization should be readily applicable to multiple other nanoparticle platforms, such as carbon nanotubes [Georgin et al., J Am Chem Soc. 131 (41): 14658-9 (2009)], quantum dots [Yang et al., Environ Health Perspect. 115 (9): 1339-43 (2007)], and iron-oxide [Jain et al., Mol Pharm. 5 (2): 316-27 (2008)] nanoparticles. These common platforms are all heavily sequestered by the reticuloendothelial system during IV administration, and could benefit from local administration. Third, multiple tumor histologies could be targeted with any of these platforms by altering the functionalized molecular target of the platform. Finally, image-guided nanoembolization is not limited to liver tumors. It can potentially be applied to any solid organ malignancy that can be accessed intra-arterially, for example and without limitation renal, pancreatic, and cranial malignancies. Nanoembolization thus offers an innovative means to deliver a broad array of nanoparticle platforms, with customizable surface-functionalized targets, to a diverse group of solid tumors.

### Example 2

The following example was performed to investigate the use of nanoembolization as a potential therapy for pancreatic cancer. Nanoembolization was shown to increase the delivery of nanoparticles to pancreatic tumors over IV delivery in a rabbit model of pancreatic cancer.

In the study, 12 rabbits were implanted with pancreatic tumors as follows. Six control animals were intravenously administered a 4:1 ration of AuNP:lipiodol composition over 3-5 minutes in a final volume of 5 mL. Tissues were then harvested and analyzed as indicated below.

The remaining six animals were intraarterially administered via nanoembolization the same composition as above following catheter (a 2F catheter was utilized) placement under fluoroscopy. The placement of the catheter was verified by TRIP-MRI, and a 4:1 ration of AuNP:lipiodol composition over 3-5 minutes in a final volume of 5 mL was administered. Tissues were then harvested and analyzed as indicated below. Results were compared using an unpaired t-test.

Gold levels in tissue samples were measured using inductively coupled plasma mass spectroscopy (ICP-MS), a very sensitive method used to quantitatively measure the amount of metals in samples. Four samples were taken from each organ that was analyzed; these samples were digested in trace metal grade, ultra pure nitric acid and then run through the ICP-MS to analyze the concentration of gold in each tissue sample. The gold concentrations in each organ were then compared between the two experimental groups (control and nanoembolization) using a student's t-test for each organ.

First, looking at just the IV levels of nanoparticles, a significant amount of uptake in the RES organs was noted, particularly the spleen and liver, compared to all other organs. Compared to the tumor, there is about 150 times more nanoparticles in the spleen. The dramatic sequestration of nanoparticles by the liver and spleen was seen which suggests why there is so little in the tumor. As can be seen in Table 2, a dramatic increase in the tumor concentration for IA delivery when compared to IV delivery was noted. Not only that, but there was a decrease in the concentrations in the liver and spleen, to about 50% in the liver and about 60% in the spleen.

Looking more closely at the increases, a dramatic increase was seen in the tumor using IA over IV—approximately 89 times as much in the tumor core, 38 times as much in the tumor periphery and 54 times as much in the tumor combined. This data is depicted in Table 3, below.

This study showed that nanoembolization greatly increases AuNP uptake in targeted organs, and further that nanoembolization reduces sequestration of AuNPs by the RES.

1. A composition comprising a nanoparticle and an embolic agent, the nanoparticle functionalized with a polynucleotide.

2. The composition of claim 1 wherein the embolic agent is selected from the group consisting of a lipid emulsion, gelatin sponge, tris acetyl gelatin microspheres, embolization coils, ethanol, small molecule drugs, biodegradable microspheres, non-biodegradable microspheres or polymers, and self-assemblying embolic material.

3. (canceled)

4. (canceled)

5. The composition of claim 1 wherein the polynucleotide is double stranded.

6. (canceled)

7. (canceled)

8. The composition of claim 1 wherein the polynucleotide comprises a detectable marker.

9. The composition of claim 1 wherein the functionalized nanoparticle and the embolic agent are present in a ratio of about 1:1 to about 10:1, a ratio of about 2:1 to about 5:1, a ratio of 3:1, a ratio of about 1:1 to about 1:10, a ratio of about 1:3 to about 1:6, or a ratio of about 1:4.

10. (canceled)

11. (canceled)

12. (canceled)

13. (canceled)

14. (canceled)

15. (canceled)

16. (canceled)

17. (canceled)

18. The composition of claim 1 further comprising a therapeutic agent.

19. (canceled)

20. (canceled)

21. A method of local delivery of the composition of claim 1 comprising the step of identifying a site for delivery and delivering the composition.

22. The method of claim 21 wherein the site is a site of pathogenesis.

23. The method of claim 22 wherein the identifying step is performed by interventional radiology.

24. The method of claim 21 wherein the delivering step is performed intraarterially or intravenously.

25. (canceled)

26. (canceled)

27. (canceled)

28. (canceled)

29. (canceled)

30. The method of claim 22 wherein the pathogenesis is associated with a cancer.

31. (canceled)

32. The method of claim 22 wherein the pathogenesis is associated with a solid organ disease.

33. (canceled)

34. The method of claim 21 wherein delivering the composition regulates the expression of a target polynucleotide.

35. The method of claim 34 wherein the target polynucleotide is survivin.

36. The method of claim 34 wherein the target polynucleotide is a microRNA (miRNA).

37. The method of claim 36 wherein the miRNA is miRNA 210.

38. The method of claim 21 wherein the site is a solid organ.

39. The method of claim 38 wherein the identifying step is performed by interventional radiology.

40. The method of claim 38 wherein the delivering step is performed intraarterially or intravenously.

41. (canceled)

42. (canceled)

43. The method of claim 38 wherein the composition regulates expression of a target polynucleotide.

44. (canceled)

45. (canceled)


SEQUENCE LISTING (1 sequence)

SEQ ID NO: 1 - DNA, 30 bp, Artificial Sequence
  Synthetic primer
cagccgtgtcacacgcacagaaaaaaaaaa